(19) United States
(12) Patent Application Publication
(10) Pub. No.: US 2026/0253401 A1
(43) Pub. Date: Aug. 27, 2026

(54) SYSTEM

(71) Applicant: SoftBank Group Corp., Tokyo (JP)

(72) Inventor: Jotaro KOIKE, Tokyo (JP)

(73) Assignee: SoftBank Group Corp., Tokyo (JP)

(21) Appl. No.: 19/536,289

(22) Filed: Feb. 11, 2026

(30) Foreign Application Priority Data
Feb. 21, 2025 (JP) 2025-027037

(51) Int. Cl. G06V 10/82; G06Q 30/02

(52) U.S. Cl. CPC G06V 10/82; G06Q 30/02

(57) ABSTRACT

The system according to the embodiment comprises a reception unit, an analysis unit, a creation unit, and a distribution unit. The reception unit receives input from a parent regarding the growth status of a child. The analysis unit analyzes information input by the reception unit. The creation unit creates a referral letter when an abnormality is detected by the analysis unit. The distribution unit distributes the referral letter created by the creation unit in PDF format.

### CROSS-REFERENCE TO RELATED APPLICATION

The present application claims priority to and incorporates by reference the entire contents of Japanese Patent Application No. 2025-027037 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that many parents are unable to participate in health checkups for children aged 1 to 3 years, making it difficult to appropriately grasp the growth status of the child.

### SUMMARY OF THE INVENTION

The system according to the embodiment comprises a reception unit, an analysis unit, a creation unit, and a distribution unit. The reception unit receives input from a parent regarding the growth status of a child. The analysis unit analyzes information input by the reception unit. The creation unit creates a referral letter when an abnormality is detected by the analysis unit. The distribution unit distributes the referral letter created by the creation unit in PDF format.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

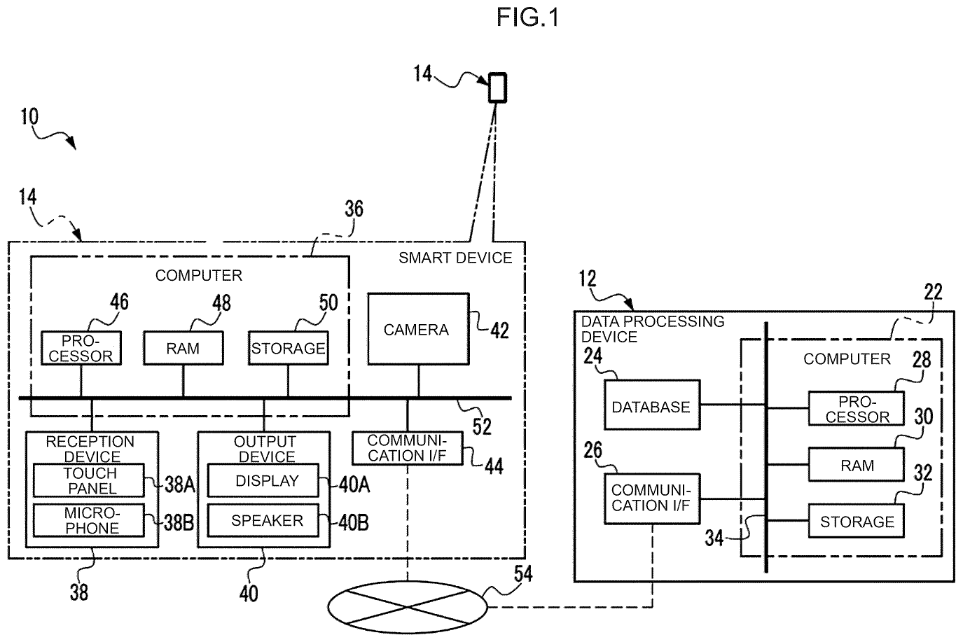

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

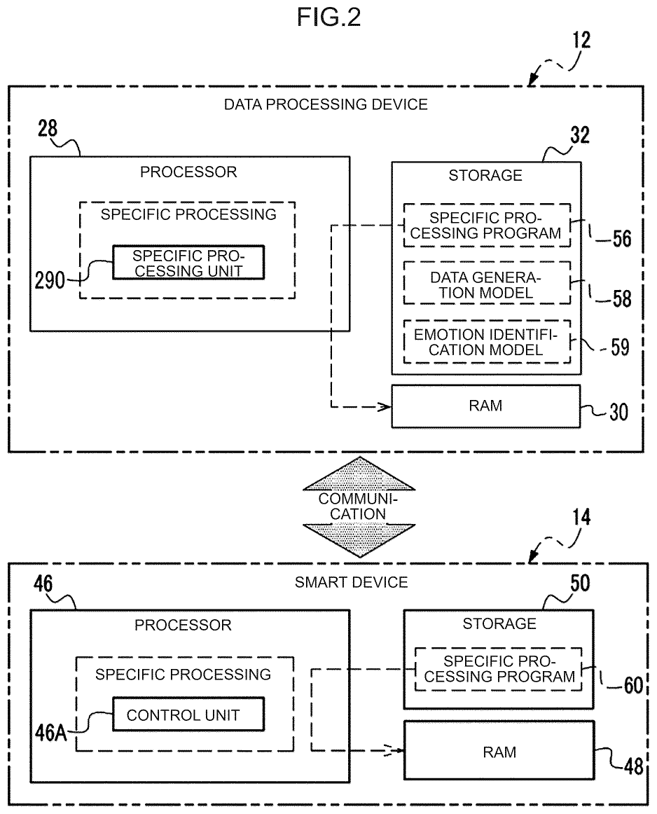

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The growth status confirmation system according to the embodiment of the present invention is a system that streamlines health checkups for children aged 1 to 3 years, enabling parents to confirm the growth status of their children without taking time off work on weekdays. This growth status confirmation system allows parents to input the growth status of their child into a generative AI, which analyzes the input based on past growth data and image data such as dental alignment. If any abnormality is detected, the system automatically creates a referral letter to the nearest hospital and distributes it in PDF format. For example, when a parent inputs the growth status of their child and the generative AI detects an abnormality and creates a referral letter, the child can receive appropriate medical care at an early stage. As a result, the growth status confirmation system enables parents to confirm their child's growth status without taking time off work on weekdays, and allows for prompt medical examination at a hospital if any abnormality is detected. Specifically, the growth status confirmation system receives growth status data input by the parent (e.g., numerical and text information such as height, weight, dental eruption, walking behavior, and image data such as dental alignment and facial appearance) at the reception unit, normalizes and vectorizes these data at the preprocessing unit, and inputs image data as, for example, 224×224 pixel RGB image tensors (shape: 224×224×3) and text data as tokenized sequence data (e.g., integer arrays of up to 512 tokens) into the generative AI. The generative AI adopts a multimodal architecture combining an image feature extraction unit using a convolutional neural network (CNN) and a text analysis unit using a transformer-based large language model (LLM), and is pre-trained using a database of past growth data (e.g., standard growth curve data by age, gender, and region, past checkup results, abnormal case images provided by medical institutions) as training data. The generative AI compares the input growth data with past data in a high-dimensional feature space and applies abnormality detection algorithms (e.g., threshold judgment, anomaly scoring, outlier detection by clustering, etc.). For example, if height or weight deviates by more than 2.5 standard deviations from the standard for age and gender, or if the CNN extracts a feature map of dental malalignment from dental alignment images, an abnormality flag is output. The output consists of structured data such as a binary label indicating the presence or absence of abnormality (0: normal, 1: abnormal), an abnormality score (continuous value from 0.0 to 1.0), and explanatory text for the abnormal site (e.g., “suspected mandibular prognathism”). These outputs are passed to the referral letter creation unit and automatically reflected in the referral letter template. The referral letter includes the child's basic information, input growth data, detected abnormality details, recommended medical departments, and information on the nearest medical institution, and the PDF generation module automatically generates a PDF file. The distribution unit distributes the PDF via means such as the parent's email address, push notifications to a dedicated app, or uploads to cloud storage. As a technical effect, this system eliminates manual visual judgment and manual creation of referral letters, and achieves high-precision and rapid abnormality detection and automatic document generation by AI, thereby streamlining checkup operations, reducing the burden on parents, and speeding up medical examinations through prior information sharing with medical institutions. Furthermore, continuous learning of the AI model enables improved judgment accuracy considering regional and individual differences, and early detection of new abnormal patterns through database expansion. Specific application fields include remote infant checkups, home health management, health monitoring at nurseries and kindergartens, and early detection support for developmental disorders and dental abnormalities.

The growth status confirmation system according to the embodiment comprises a reception unit, an analysis unit, a creation unit, and a distribution unit. The reception unit receives input from a parent regarding the growth status of a child. When the parent inputs the growth status of the child, information such as height, weight, dental eruption, and walking behavior can be entered. The reception unit can receive information via methods such as text input or voice input. The analysis unit analyzes the information input by the reception unit using a generative AI. The generative AI has learned past growth data of children and image data such as dental alignment, and checks for abnormalities by comparing the input information. For example, the generative AI detects cases where height or weight significantly deviates from the average, or where abnormalities are observed in dental alignment. The creation unit creates a referral letter when an abnormality is detected by the analysis unit. The referral letter includes the child's growth status and details of the abnormality, and is distributed in PDF format. The distribution unit distributes the referral letter created by the creation unit in PDF format. By bringing this referral letter to the hospital, the parent can receive prompt and appropriate medical examination. As a result, the growth status confirmation system according to the embodiment enables parents to confirm their child's growth status without taking time off work on weekdays, and allows for prompt medical examination at a hospital if any abnormality is detected. Specifically, the growth status confirmation system assumes that the reception unit receives growth status data from the parent, such as numerical data (e.g., height 90 cm, weight 13 kg), text data (e.g., “eight front teeth have erupted,” “unsteady walking”), and image data (e.g., oral cavity images or facial photos taken with a smartphone) regarding dental alignment and facial appearance. After receiving these data, the reception unit performs normalization and vectorization at the preprocessing unit. For example, image data is input as a 224×224 pixel RGB image tensor (shape: 224×224×3), text data as an integer array of up to 512 tokens, and voice input is converted to text by a speech recognition engine and then transformed into sequence data, with all data converted into a unified data structure. The analysis unit adopts a multimodal AI architecture combining an image feature extraction unit using a convolutional neural network (CNN) and a text analysis unit using a transformer-based large language model (LLM). The analysis unit compares the input growth data with a database of past growth data (e.g., standard growth curves by age, gender, and region, past checkup results, abnormal case images provided by medical institutions) in a high-dimensional feature space and applies abnormality detection algorithms (e.g., threshold judgment by standard deviation, anomaly scoring, outlier detection by clustering, etc.). Examples of AI input include (1) numerical vectors for height, weight, age, and gender (e.g., [90, 13, 2, 0]), (2) dental alignment image tensors, and (3) text sequences describing walking behavior (e.g., “unsteady walking”). Examples of AI output include (1) binary label indicating the presence or absence of abnormality (0: normal, 1: abnormal), (2) abnormality score (continuous value from 0.0 to 1.0), and (3) explanatory text for the abnormal site (e.g., “suspected mandibular prognathism”). The analysis unit passes these outputs to the creation unit, which automatically reflects them in the referral letter template. The creation unit includes the child's basic information, input growth data, detected abnormality details, recommended medical departments, and information on the nearest medical institution in the referral letter, and generates a PDF file using a PDF generation module. The distribution unit distributes the PDF via means such as the parent's email address, push notifications to a dedicated app, or uploads to cloud storage. As a technical effect, this system eliminates manual visual judgment and manual creation of referral letters, and achieves high-precision and rapid abnormality detection and automatic document generation by AI, thereby streamlining checkup operations, reducing the burden on parents, and speeding up medical examinations through prior information sharing with medical institutions. Furthermore, continuous learning of the AI model enables improved judgment accuracy considering regional and individual differences, and early detection of new abnormal patterns through database expansion. Specific application fields include remote infant checkups, home health management, health monitoring at nurseries and kindergartens, and early detection support for developmental disorders and dental abnormalities.

The growth status confirmation system comprises a learning unit configured to specify the type and scope of data to be learned by the generative AI. The learning unit specifies the type and scope of data to be learned by the generative AI. For example, the learning unit can specify text data, image data, or data from a specific period. By specifying the type and scope of data to be learned by the generative AI, the accuracy of analysis is improved. Some or all of the above-described processing in the learning unit may be performed using AI or without using AI. For example, the learning unit can input past growth data into the generative AI and cause the generative AI to perform data learning. Specifically, the growth status confirmation system has a function in which the learning unit classifies and selects growth data sets obtained from a database (e.g., standard growth curves by age, gender, and region, past checkup results, abnormal case images provided by medical institutions, lifestyle data collected from parents) by data type and explicitly specifies them as learning targets for the generative AI. For image data, the learning unit can selectively specify multiple image formats, such as 224×224 pixel RGB image tensors (shape: 224×224×3) or 512×512 pixel grayscale image tensors. For text data, the learning unit can specify tokenized sequence data of up to 512 tokens or paragraph-level data of growth records described in natural language. For period specification, time-series filtering can be applied, such as “only data from the past year” or “only abnormal cases from the past five years.” The learning unit passes these specification details as metadata to the learning pipeline of the generative AI, which uses only the specified data as input for batch learning or online learning. Examples of AI input include (1) a set of height, weight, and dental alignment images of two-year-olds from 2022 to 2023, (2) frame sequences of walking videos of three-year-olds from a specific region, and (3) text record data extracted only for abnormal cases. Examples of AI output include (1) feature extraction parameters for each data type, (2) weight matrices of abnormality detection models, and (3) accuracy evaluation scores of trained models (e.g., AUC=0.95, F1=0.92). The learning unit links these outputs to the analysis unit and creation unit, contributing to improved accuracy in subsequent abnormality detection and referral letter creation. The learning unit can switch between multiple learning methods, such as supervised learning, self-supervised learning, and transfer learning, and can perform multimodal learning, such as simultaneous learning of image feature extraction by CNN and text understanding models based on Transformer. The learning unit also has advanced preprocessing and postprocessing functions, such as quality control of learning data (e.g., noise removal, annotation accuracy verification), data augmentation (e.g., image rotation and flipping, text paraphrase generation), and prevention of overfitting using validation sets. As a technical effect, the learning unit realizes automatic management and optimization of large-scale and diverse datasets that are difficult to achieve with manual data selection and labeling by humans, dramatically improving the learning efficiency and analysis accuracy of the generative AI. As a result, compared to conventional rule-based or single data type-dependent systems, reproducibility, versatility, and new pattern discovery capability in abnormality detection are greatly enhanced. Specific application fields include automation of infant checkups, health monitoring considering regional characteristics, construction of case databases for medical institutions, and promotion of medical DX through continuous updates of AI models.

The growth status confirmation system comprises a format unit configured to specify the format of information input by the parent. The format unit specifies the format of information input by the parent. For example, the format unit can specify the type of input items and input formats (text, numerical, etc.). By specifying the format of information input by the parent, input efficiency is improved. Some or all of the above-described processing in the format unit may be performed using AI or without using AI. For example, the format unit can input the format of information input by the parent into the generative AI and cause the generative AI to execute the format specification. Specifically, the growth status confirmation system has a function in which the format unit defines in detail the types of input items (e.g., height, weight, dental eruption, walking behavior, facial photo, oral cavity image), input formats (e.g., numerical input, text input, image upload, voice input), and input constraints (e.g., numerical range, required items, maximum number of input characters) for the parent's input interface, and dynamically instructs these to the generative AI. For example, the format unit inputs format specifications for each item as metadata into the generative AI, such as “height: numerical (cm, 70-120)”, “walking behavior: text (up to 100 characters)”, “dental alignment image: JPEG/PNG format, up to 5 MB”. Examples of AI input include (1) format specification “height: numerical, weight: numerical, dental alignment: image, walking: text”, (2) input constraint specification “voice input only allowed, image upload required”, and (3) layout specification “input item order: height→weight→image→text”. Examples of AI output include (1) HTML/JSON structure of input forms conforming to the specified format, (2) input error detection rules (e.g., warning for out-of-range values), and (3) input assistance messages (e.g., “The image is blurry. Please retake.”). The format unit can also automatically generate and optimize the optimal input format according to the parent's input history and device characteristics (e.g., smartphone, tablet, PC). Furthermore, the format unit realizes advanced interaction control, such as real-time validation during input and dynamic addition or deletion of items according to input content (e.g., displaying additional questions only when dental abnormality is suspected). As a technical effect, the format unit automatically provides an optimized input experience for each user and situation, which was difficult to achieve with conventional static input forms or manual design by humans, thereby reducing input errors, standardizing data quality, and streamlining input operations. As a result, the accuracy of growth data collection is improved, and the reliability of subsequent AI analysis and referral letter creation is greatly enhanced. Specific application fields include automatic UI generation for infant checkup apps, input assistance for home health management systems, and electronic and automatic optimization of medical questionnaires for medical institutions.

The growth status confirmation system comprises an algorithm unit configured to specify an algorithm to be used by the generative AI during analysis. The algorithm unit specifies the algorithm to be used by the generative AI during analysis. For example, the algorithm unit can specify machine learning algorithms or statistical methods. By specifying the algorithm to be used by the generative AI during analysis, the accuracy of analysis is improved. Some or all of the above-described processing in the algorithm unit may be performed using AI or without using AI. For example, the algorithm unit can input the analysis algorithm into the generative AI and cause the generative AI to execute the algorithm specification. Specifically, the growth status confirmation system has a function in which the algorithm unit specifies in detail to the analysis unit the architecture of the AI model to be used (e.g., convolutional neural network, recurrent neural network, transformer, decision tree, random forest, support vector machine, etc.), learning methods (e.g., supervised learning, unsupervised learning, semi-supervised learning, reinforcement learning), abnormality detection algorithms (e.g., threshold judgment, anomaly scoring, clustering, reconstruction error judgment by autoencoder, etc.), and analysis parameters (e.g., threshold values, number of clusters, type of loss function, optimization algorithm, etc.). For example, the algorithm unit inputs the overall configuration of the analysis pipeline as metadata into the generative AI, such as “use CNN for image analysis, Transformer for text analysis, and Isolation Forest for anomaly detection”, “determine abnormality for deviations of 2.5 standard deviations or more”, “use cross-entropy for the loss function, Adam for optimization”, etc. Examples of AI input include (1) algorithm specification “image: CNN (ResNet architecture), text: Transformer (12 layers), anomaly detection: One-Class SVM”, (2) parameter specification “threshold=2.0, number of clusters=5, loss function=MSE”, and (3) learning method specification “supervised learning+transfer learning”. Examples of AI output include (1) analysis results based on the specified algorithm (e.g., anomaly score 0.87, abnormal site “mandibular prognathism”), (2) accuracy evaluation for each algorithm (e.g., AUC=0.93, Recall=0.90), and (3) model weight files after parameter optimization. The algorithm unit can automatically control hybrid analysis combining multiple algorithms or sequential algorithm switching (e.g., threshold judgment first, then clustering) according to the analysis target and data characteristics. Furthermore, the algorithm unit can automatically readjust parameters based on feedback from analysis results to continuously improve accuracy. As a technical effect, the algorithm unit realizes automatic design and optimization of the optimal AI analysis pipeline according to data characteristics and analysis objectives, which was difficult to achieve with manual algorithm selection or static rule-based processing by humans, thereby greatly improving analysis accuracy, reproducibility, and versatility. As a result, early detection of growth abnormalities, support for personalized medicine, and standardization of analysis methods among medical institutions become possible. Specific application fields include automatic abnormality detection in infant checkups, dental image diagnosis support, developmental disorder screening, and medical big data analysis platforms.

The reception unit is configured to estimate the parent's emotion and adjust the design of the input interface based on the estimated emotion. For example, if the parent is feeling stressed, the reception unit provides a simple and intuitive interface and minimizes the input steps. If the parent is relaxed, the reception unit can provide detailed input options and propose customizable input methods. Furthermore, if the parent is in a hurry, the reception unit can prioritize voice input to enable rapid input of the child's growth status. By adjusting the design of the input interface according to the parent's emotion, the burden on the parent is reduced. Emotion estimation is realized using, for example, an emotion engine or generative AI with emotion estimation functions. The generative AI may be a text generative AI (e.g., LLM) or a multimodal generative AI, but is not limited to these examples. Some or all of the above-described processing in the reception unit may be performed using AI or without using AI. For example, the reception unit can input the parent's emotion data into the generative AI and cause the generative AI to perform emotion estimation. Specifically, the reception unit inputs text data entered by the parent (e.g., natural language sentences such as “I am busy today and have no time” or “Input is difficult”), voice data (e.g., audio waveform data, spectrogram), and facial image data (e.g., facial expression images taken with a smartphone camera, 224×224 pixel RGB image tensor) into the emotion estimation AI. Examples of AI input include (1) text sequences such as “Input is troublesome” (integer array of up to 512 tokens), (2) mel spectrogram of the parent's speech (shape: 128×256), and (3) facial expression image tensor (shape: 224×224×3). The reception unit normalizes and extracts features from these input data at the preprocessing unit and inputs them into a multimodal emotion estimation AI (e.g., integrated CNN+Transformer model). The AI classifies emotion categories (e.g., stress, relaxation, hurry, enjoyment) from the input data and generates output such as emotion label (e.g., “stress”), emotion score (continuous value from 0.0 to 1.0), and explanatory text for the emotion estimation (e.g., “Judged as stress due to fast speech and tense facial expression”). Examples of AI output include (1) emotion label “stress”, (2) emotion score 0.82, and (3) explanation “The word ‘busy’ is included in the input sentence”. Based on these outputs, the reception unit instructs the interface generation module to automatically generate UI design parameters, such as “limit input items to three”, “place the voice input button at the top”, and “change the color scheme to a calm blue tone”. Furthermore, the reception unit reconstructs the interface in real time when the parent's emotional state changes, maintaining the optimal UI even during input. As a technical effect, the reception unit achieves high-precision emotion estimation and dynamic UI optimization by AI, without relying on human subjective judgment or static UI design, thereby greatly reducing the psychological burden of input work and decreasing input errors and dropout rates. The system automates user-specific optimization, which was difficult with conventional uniform UIs, and improves overall system usability and data quality. Specific application fields include personalized UI for infant checkup apps, stress-responsive input support for home health management systems, and emotion-adaptive electronic medical questionnaires for medical institutions.

The reception unit is configured to analyze the parent's past input history and propose an appropriate input method. For example, the reception unit automatically displays growth data that the parent has frequently entered in the past as candidates. The reception unit can also preferentially propose input methods (voice, text, etc.) that the parent has used in the past. Furthermore, the reception unit can predict and propose growth data to be used at specific times based on the parent's past input history. By analyzing the parent's past input history, the reception unit can propose the optimal input method. Some or all of the above-described processing in the reception unit may be performed using AI or without using AI. For example, the reception unit can input the parent's past input history data into the generative AI and cause the generative AI to propose the optimal input method. Specifically, the reception unit inputs a database of input history accumulated for each parent (e.g., structured data such as input date and time, input items, input values, input methods, input device information for the past year) as time-series vectors (e.g., vectorizing each history entry, shape: N×M, N is the number of histories, M is the number of features) into the AI. Examples of AI input include (1) history sequences such as “2023/05/01 20:00, height, 90 cm, text, smartphone”, “2023/05/08 21:00, weight, 13 kg, voice, smartphone”, (2) time-series array of input frequency (e.g., number of inputs per week), and (3) category distribution of input methods (e.g., text 70%, voice 30%). The reception unit inputs these history data into a time-series analysis AI based on LSTM or Transformer and extracts input patterns for each parent. The AI predicts the input method and input items most likely to be used at the next input based on the input history, and generates output such as recommended input method label (e.g., “voice input recommended”), candidate input item list (e.g., “height”, “weight”), and recommendation reason text (e.g., “voice input used three times in a row”). Examples of AI output include (1) recommended input method “voice”, (2) candidate items “height”, “weight”, and (3) recommendation reason “voice input usage rate is high at night”. Based on these outputs, the reception unit automatically generates the input interface and immediately presents the optimal input method and candidate items when the parent opens the input screen. Furthermore, the reception unit monitors the parent's input behavior in real time and dynamically switches input methods and updates candidate items. As a technical effect, the reception unit automates personalized input support based on user input tendencies using AI, which was difficult with human history analysis or static candidate presentation, thereby streamlining input work, reducing input errors, and improving user satisfaction. Specific application fields include history-learning input support for infant checkup apps, personalized input forms for home health management systems, and automatic candidate presentation for electronic medical questionnaires for medical institutions.

The reception unit is configured to customize input items at the time of input based on the parent's current living situation and areas of interest. For example, if the parent is busy, the reception unit prompts input of only the most important growth data. If the parent is interested in health, the reception unit prompts input of detailed health data. Furthermore, if the parent is interested in education, the reception unit prompts input of education-related growth data. By customizing input items according to the parent's living situation and areas of interest, input efficiency is improved. Some or all of the above-described processing in the reception unit may be performed using AI or without using AI. For example, the reception unit can input data on the parent's living situation and areas of interest into the generative AI and cause the generative AI to customize input items. Specifically, the reception unit inputs living situation data entered by the parent (e.g., text or numerical data such as occupation, working hours, family structure, health status, hobbies, educational policy) and areas of interest data (e.g., category labels such as “health”, “education”, “sports”) as structured vectors (e.g., one-hot encoding for each category, normalized values for numerical data) into the AI. Examples of AI input include (1) “Occupation: company employee, working hours: 9-18, interest: health”, (2) “Family structure: parents+one child, hobby: sports, interest: education”, and (3) “Health status: good, interest: development”. The reception unit inputs these data into a Transformer-based feature extraction AI and infers the optimal set of input items for the parent's living situation and areas of interest. The AI calculates importance scores from the input data and generates output such as input item list (e.g., “height”, “weight”, “dietary content”), priority for each item (e.g., importance score 0.95), and customization reason text (e.g., “Added dietary content due to high health interest”). Examples of AI output include (1) input items “height”, “weight”, “dietary content”, (2) priority “height: 0.95, weight: 0.90, dietary content: 0.85”, and (3) reason “high health interest”. Based on these outputs, the reception unit automatically generates the input form and adds, deletes, or reorders items in real time according to the parent's situation. Furthermore, the reception unit immediately reconstructs input items when the parent's living situation or areas of interest change, always providing the optimal input experience. As a technical effect, the reception unit automates dynamic optimization of input items according to each user's situation and interests using AI, which was difficult with manual customization or static input item design by humans, thereby streamlining input work, standardizing data quality, and improving user satisfaction. Specific application fields include lifestyle-adaptive input support for infant checkup apps, data collection by areas of interest for home health management systems, and personalized automatic generation of medical questionnaires for medical institutions.

The reception unit is configured to estimate the parent's emotion and determine the priority of input based on the estimated emotion. For example, if the parent is feeling stressed, the reception unit preferentially displays the most important input items. If the parent is relaxed, the reception unit can sequentially display detailed input items. Furthermore, if the parent is in a hurry, the reception unit can prioritize voice input to enable rapid input. By determining the priority of input according to the parent's emotion, input efficiency is improved. Emotion estimation is realized using, for example, an emotion engine or generative AI with emotion estimation functions. The generative AI may be a text generative AI (e.g., LLM) or a multimodal generative AI, but is not limited to these examples. Some or all of the above-described processing in the reception unit may be performed using AI or without using AI. For example, the reception unit can input the parent's emotion data into the generative AI and cause the generative AI to perform emotion estimation. Specifically, the reception unit inputs text data entered by the parent (e.g., natural language sentences such as “I am tired today”, “I am in a hurry”), voice data (e.g., audio waveform, mel spectrogram), and facial image data (e.g., facial expression image tensor) into the emotion estimation AI. Examples of AI input include (1) text sequences such as “I am in a hurry” (up to 512 tokens), (2) spectrogram of the parent's speech (shape: 128×256), and (3) facial expression image tensor (shape: 224×224×3). The reception unit inputs these data into a multimodal AI (CNN+Transformer) and classifies emotion categories (e.g., stress, relaxation, hurry). The AI generates output such as emotion label (e.g., “hurry”), emotion score (0.0-1.0), and explanatory text for the estimation (e.g., “Speech rate is fast”). Examples of AI output include (1) emotion label “hurry”, (2) emotion score 0.91, and (3) explanation “The word ‘hurry’ is included in the input sentence”. Based on these outputs, the reception unit determines the priority of input items, for example, displaying only important items such as “height” and “weight” first and postponing detailed items. UI optimization such as placing the voice input button at the top is also performed. Furthermore, the reception unit reconstructs the order of input items in real time when the parent's emotional state changes, always providing the optimal input experience. As a technical effect, the reception unit achieves high-precision emotion estimation and dynamic optimization of input items by AI, without relying on human subjective judgment or static input item design, thereby streamlining input work, reducing input errors, and improving user satisfaction. Specific application fields include emotion-adaptive input support for infant checkup apps, stress-responsive input optimization for home health management systems, and emotion-linked automatic generation of medical questionnaires for medical institutions.

The reception unit is configured to preferentially display highly relevant input items at the time of input by considering the parent's geographic location information. For example, if the parent lives in a specific region, the reception unit preferentially displays growth data relevant to that region. If the parent is traveling, the reception unit can preferentially display growth data relevant to the travel destination. Furthermore, if the parent is planning to move, the reception unit can preferentially display growth data relevant to the new region. By displaying input items based on the parent's geographic location information, input efficiency is improved. Some or all of the above-described processing in the reception unit may be performed using AI or without using AI. For example, the reception unit can input the parent's geographic location information data into the generative AI and cause the generative AI to display highly relevant input items. Specifically, the reception unit normalizes GPS coordinate data obtained from the parent's device (e.g., latitude and longitude numerical vectors, e.g., 35.6895, 139.6917), region codes linked to location information (e.g., prefecture, city/ward/town/village ID), and movement history (e.g., time-series vector of location information for the past week, shape: 7×2) at the preprocessing unit and inputs them into the AI. Examples of AI input include (1) “Shinjuku-ku, Tokyo, current location: 35.6938, 139.7034”, (2) “Travel destination: Kita-ku, Osaka, movement history: Tokyo→Osaka”, and (3) “Planned move: Nishi-ku, Yokohama” as structured data. The reception unit inputs these geographic information into a Transformer-based location information analysis AI and infers region-specific growth data items (e.g., region-specific vaccination schedules, region-specific infectious disease risks, health management items according to climate). The AI calculates relevance scores from the input geographic information and generates output such as a list of input items to be preferentially displayed (e.g., “influenza vaccination”, “hay fever measures”, “heat stroke risk”), priority for each item (e.g., importance score 0.92), and region-specific advice text (e.g., “Hay fever is prevalent in this region in spring”). Examples of AI output include (1) input items “influenza vaccination”, “hay fever measures”, (2) priority “influenza vaccination: 0.92, hay fever measures: 0.85”, and (3) advice “Be careful of hay fever in spring”. Based on these outputs, the reception unit automatically generates the input form and adds, deletes, or reorders items in real time according to the parent's current location or planned movement. Furthermore, the reception unit immediately reconstructs input items when the parent's location information changes, always providing the optimal input experience. As a technical effect, the reception unit automates dynamic optimization of input items according to the user's geographic situation using AI, which was difficult with manual customization or static input item design by humans, thereby streamlining input work, standardizing data quality, and improving user satisfaction. The system can flexibly respond to region-specific health risks and differences in medical systems that could not be addressed with conventional uniform input forms, thereby improving the accuracy of growth data collection and greatly enhancing the reliability of subsequent AI analysis and referral letter creation. Specific application fields include region-adaptive input support for infant checkup apps, region-specific health monitoring for home health management systems, region-specific automatic generation of medical questionnaires for medical institutions, and region-specific health risk input support during disasters.

The reception unit is configured to analyze the parent's social media activity at the time of input and propose relevant input items. For example, the reception unit proposes relevant input items based on growth data of the child shared by the parent on social media. The reception unit can also propose relevant input items based on health-related accounts followed by the parent on social media. Furthermore, the reception unit can propose relevant input items based on information from parenting groups in which the parent participates on social media. By analyzing the parent's social media activity, the reception unit can propose relevant input items. Some or all of the above-described processing in the reception unit may be performed using AI or without using AI. For example, the reception unit can input the parent's social media activity data into the generative AI and cause the generative AI to propose relevant input items. Specifically, the reception unit stores post data obtained from multiple social media platforms used by the parent (e.g., text posts, image posts, video links), followed account lists (e.g., arrays of health and parenting-related account IDs), and group participation information (e.g., parenting group name, activity frequency, summary of post content) in a structured database, performs natural language processing, image analysis, and category classification at the preprocessing unit, and inputs them into the AI. Examples of AI input include (1) “2023/06/01 post: My child's teeth have started to erupt (image attached)”, (2) “Followed accounts: official pediatrician, baby food advisor” as text and image data, and (3) “Participating group: 1-year-old moms' group, posts once a week” as group activity data. The reception unit inputs these data into a Transformer-based multimodal AI and infers highly relevant growth data items (e.g., dental eruption, baby food content, developmental stage checklist) from the parent's areas of interest, recent topics, and post content. The AI extracts features from the input data and generates output such as a list of input items to be proposed (e.g., “dental eruption”, “baby food content”, “speech status”), priority for each item (e.g., importance score 0.88), and proposal reason text (e.g., “Recent posts mention dental eruption”). Examples of AI output include (1) input items “dental eruption”, “baby food content”, (2) priority “dental eruption: 0.88, baby food content: 0.82”, and (3) reason “Followed account is related to baby food”. Based on these outputs, the reception unit automatically generates the input form and adds, deletes, or reorders items in real time according to the parent's social media activity. Furthermore, the reception unit immediately reconstructs input items when the parent makes new posts or changes group participation status, always providing the optimal input experience. As a technical effect, the reception unit automates dynamic optimization of input items according to the user's online activity using AI, which was difficult with manual customization or static input item design by humans, thereby streamlining input work, standardizing data quality, and improving user satisfaction. The system can flexibly respond to individual interests and the latest topics that could not be addressed with conventional uniform input forms, thereby improving the accuracy of growth data collection and greatly enhancing the reliability of subsequent AI analysis and referral letter creation. Specific application fields include SNS-linked input support for infant checkup apps, automatic extraction of areas of interest for home health management systems, and online activity-linked automatic generation of medical questionnaires for medical institutions.

The analysis unit is configured to estimate the parent's emotion and adjust the display method of the analysis result based on the estimated emotion. For example, if the parent is nervous, the analysis unit provides a simple and highly visible display method. If the parent is relaxed, the analysis unit can provide a display method including detailed information. Furthermore, if the parent is in a hurry, the analysis unit can provide a display method that focuses on key points. By adjusting the display method of the analysis result according to the parent's emotion, the parent's understanding is deepened. Emotion estimation is realized using, for example, an emotion engine or generative AI with emotion estimation functions. The generative AI may be a text generative AI (e.g., LLM) or a multimodal generative AI, but is not limited to these examples. Some or all of the above-described processing in the analysis unit may be performed using AI or without using AI. For example, the analysis unit can input the parent's emotion data into the generative AI and cause the generative AI to perform emotion estimation. Specifically, the analysis unit inputs text data entered by the parent (e.g., natural language sentences such as “I am worried about the result”, “I will check the details later”), voice data (e.g., audio waveform data, mel spectrogram), and facial image data (e.g., facial expression image tensor, shape: 224×224×3) into the emotion estimation AI. The analysis unit normalizes and extracts features from these data at the preprocessing unit and inputs them into a multimodal emotion estimation AI integrating CNN and Transformer. Examples of AI input include (1) text sequences such as “I will check the details later” (up to 512 tokens), (2) mel spectrogram of the parent's speech (shape: 128×256), and (3) facial expression image tensor (shape: 224×224×3). The analysis unit receives output from the AI such as emotion label (e.g., “nervous”, “relaxed”, “in a hurry”), emotion score (continuous value from 0.0 to 1.0), and explanatory text for the estimation (e.g., “Judged as nervous due to fast speech and tense facial expression”). Based on these outputs, the analysis unit instructs the display control module, for example, to emphasize only important items in large font and fold detailed information for the “nervous” label, to add detailed graphs, comparisons with past data, and explanations of abnormality detection for the “relaxed” label, and to display only key points in bullet points and shorten the overall display time for the “in a hurry” label. The analysis unit reconstructs the display method in real time when the parent's emotional state changes, always maintaining optimal information presentation. As a technical effect, the analysis unit achieves high-precision emotion estimation and dynamic UI optimization by AI, without relying on human subjective judgment or static UI design, thereby improving understanding of analysis results, preventing confusion due to information overload, and improving user satisfaction through user-specific optimization. The system can flexibly respond to user-specific information needs, which was difficult with conventional uniform displays, and also contributes to accountability for analysis results and support for informed consent in medical settings. Specific application fields include emotion-adaptive result display for infant checkup apps, stress-responsive information presentation for home health management systems, and personalized result explanation for electronic medical records for medical institutions.

The analysis unit is configured to optimize the analysis algorithm by referring to past growth data during analysis. For example, the analysis unit selects the optimal analysis algorithm based on past growth data. The analysis unit can also analyze past growth data and adjust the parameters of the analysis algorithm. Furthermore, the analysis unit can refer to past growth data to improve the accuracy of the analysis algorithm. By referring to past growth data, the accuracy of the analysis algorithm is improved. Some or all of the above-described processing in the analysis unit may be performed using AI or without using AI. For example, the analysis unit can input past growth data into the generative AI and cause the generative AI to optimize the analysis algorithm. Specifically, the analysis unit inputs a database of past growth data (e.g., standard growth curves by age, gender, and region, past checkup results, abnormal case images, lifestyle data) as time-series vectors (shape: N×M, N is the number of data entries, M is the number of features), image tensors (e.g., 224×224×3), and text sequences (up to 512 tokens) into the AI. Examples of AI input include (1) a set of height, weight, and dental alignment images of two-year-olds over the past five years, (2) text record data extracted only for abnormal cases, and (3) region-specific growth curve data vectors. The analysis unit inputs these data into the AI using methods such as supervised learning, transfer learning, and self-supervised learning, and automatically selects the optimal algorithm from multiple algorithms such as CNN, Transformer, decision tree, and random forest. The AI performs cross-validation and grid search on the input data and generates output such as the name of the optimal algorithm (e.g., “CNN+Isolation Forest”), parameter set (e.g., “threshold=2.5, number of clusters=4”), and accuracy evaluation score (e.g., AUC=0.96, F1=0.93). Based on these outputs, the analysis unit automatically configures the analysis pipeline and strengthens reproducibility, versatility, and new abnormal pattern discovery capability of the analysis results. Furthermore, the analysis unit continuously readjusts parameters based on feedback from analysis results to improve model accuracy. As a technical effect, the analysis unit realizes automatic design and optimization of the optimal AI analysis pipeline according to data characteristics and analysis objectives, which was difficult to achieve with manual algorithm selection or static rule-based processing by humans, thereby greatly improving analysis accuracy, reproducibility, and versatility. As a result, early detection of growth abnormalities, support for personalized medicine, and standardization of analysis methods among medical institutions become possible. Specific application fields include automatic abnormality detection in infant checkups, dental image diagnosis support, developmental disorder screening, and medical big data analysis platforms.

The analysis unit is configured to apply different analysis methods according to the category of the child (age, gender, etc.) during analysis. For example, the analysis unit applies the optimal analysis method based on growth data according to age. The analysis unit can also apply the optimal analysis method based on growth data according to gender. Furthermore, the analysis unit can apply the optimal analysis method based on growth data according to the child's specific health condition. By applying analysis methods according to the category of the child, the accuracy of analysis is improved. Some or all of the above-described processing in the analysis unit may be performed using AI or without using AI. For example, the analysis unit can input category data of the child into the generative AI and cause the generative AI to apply analysis methods. Specifically, the analysis unit inputs category data such as age, gender, and health condition of the child (e.g., age=2 years, gender=male, health condition=allergy present) as one-hot encoding or numerical vectors into the AI. Examples of AI input include (1) category vector for age, gender, and health condition (e.g., [2, 0, 1]), (2) age-specific growth curve data, and (3) gender-specific standard value data. Based on these inputs, the analysis unit automatically selects and configures the analysis pipeline, such as applying CNN for image analysis by age, parametric models by gender, and abnormality detection algorithms (Isolation Forest, One-Class SVM, etc.) by health condition. The AI generates output such as the name of the applied analysis method (e.g., “2-year-old: CNN+threshold judgment”, “female: standard deviation scoring”), analysis result (e.g., anomaly score 0.85), and reason text for method selection (e.g., “Applied CNN based on the standard growth curve for 2-year-olds”). Based on these outputs, the analysis unit structures the analysis results and realizes optimal abnormality detection and growth judgment for each child. As a technical effect, the analysis unit automates individual optimization by AI, which was difficult with conventional uniform algorithm application, thereby greatly improving judgment accuracy, explainability, and user satisfaction. Specific application fields include individual optimization for infant checkups, developmental disorder screening, gender-specific medical support, and risk assessment by health condition.

The analysis unit is configured to estimate the parent's emotion and determine the priority of the analysis result based on the estimated emotion. For example, if the parent is feeling stressed, the analysis unit preferentially displays the most important analysis results. If the parent is relaxed, the analysis unit can sequentially display detailed analysis results. Furthermore, if the parent is in a hurry, the analysis unit can preferentially display analysis results that focus on key points. By determining the priority of the analysis result according to the parent's emotion, important information can be provided preferentially. Emotion estimation is realized using, for example, an emotion engine or generative AI with emotion estimation functions. The generative AI may be a text generative AI (e.g., LLM) or a multimodal generative AI, but is not limited to these examples. Some or all of the above-described processing in the analysis unit may be performed using AI or without using AI. For example, the analysis unit can input the parent's emotion data into the generative AI and cause the generative AI to perform emotion estimation. Specifically, the analysis unit inputs text data entered by the parent (e.g., natural language sentences such as “I am in a hurry”, “Details later”), voice data (e.g., audio waveform, mel spectrogram), and facial image data (e.g., facial expression image tensor) into the emotion estimation AI. Examples of AI input include (1) text sequences such as “I am in a hurry” (up to 512 tokens), (2) spectrogram of the parent's speech (shape: 128×256), and (3) facial expression image tensor (shape: 224×224×3). The analysis unit inputs these data into an integrated CNN+Transformer model and classifies emotion categories (e.g., stress, relaxation, hurry). The AI generates output such as emotion label (e.g., “hurry”), emotion score (0.0-1.0), and explanatory text for the estimation (e.g., “The word ‘hurry’ is included in the input sentence”). Based on these outputs, the analysis unit determines the priority of the analysis results, for example, displaying important information such as “presence or absence of abnormality” and “items to watch out for” first, and postponing detailed graphs and explanations. Furthermore, when the parent's emotional state changes, the analysis unit reconstructs the display order in real time, always maintaining optimal information presentation. As a technical effect, the analysis unit achieves high-precision emotion estimation and dynamic priority control by AI, without relying on human subjective judgment or static display order design, thereby preventing important information from being overlooked, improving user satisfaction, and streamlining input work. Specific application fields include emotion-adaptive result presentation for infant checkup apps, stress-responsive information prioritization for home health management systems, and personalized result display for electronic medical records for medical institutions.

The analysis unit is configured to weight the analysis based on the submission timing of the child's growth data during analysis. For example, the analysis unit places greater emphasis on recently submitted growth data during analysis. The analysis unit can also weight the analysis based on growth data submitted during a specific period. Furthermore, the analysis unit can adjust the importance of growth data according to the submission timing and perform analysis. By weighting the analysis based on the submission timing of the child's growth data, the accuracy of analysis is improved. Some or all of the above-described processing in the analysis unit may be performed using AI or without using AI. For example, the analysis unit can input submission timing data of growth data into the generative AI and cause the generative AI to perform weighting of the analysis. Specifically, the analysis unit vectorizes time-series data such as submission date and time of growth data (e.g., UNIX timestamp, YYYY/MM/DD format), submission interval, and submission frequency, and inputs them into the AI. Examples of AI input include (1) history sequences such as “2023/05/01 20:00, height, 90 cm”, “2023/06/01 20:00, height, 91 cm”, (2) time-series array of submission intervals (e.g., 30 days, 31 days), and (3) statistical values of submission frequency (e.g., twice a month). The analysis unit inputs these data into a time-series analysis AI based on LSTM or Transformer and generates a weighting vector that assigns higher weights to the latest data or data from specific periods. The AI generates output such as a weighted growth data set, list of weight values (e.g., latest data=1.0, one year ago=0.5), and explanatory text for weighting (e.g., “Increased weight due to higher reliability of latest data”). Based on these outputs, the analysis unit applies weight parameters to the analysis algorithm and improves the accuracy of abnormality detection and growth judgment. As a technical effect, the analysis unit automates dynamic optimization of time-series data weighting by AI, which was difficult with uniform weighting or manual adjustment by humans, thereby reflecting the latest information, controlling the influence of past data, and improving analysis accuracy. Specific application fields include time-series growth analysis for infant checkups, latest data-focused judgment for health management systems, and time-dependent abnormality detection for medical institutions.

The analysis unit is configured to improve the accuracy of analysis by referring to relevant medical literature during analysis. For example, the analysis unit refers to the latest medical literature to optimize the analysis algorithm. The analysis unit can also improve the accuracy of analysis results based on relevant medical literature. Furthermore, the analysis unit can incorporate medical literature data into the analysis to provide more accurate results. By referring to relevant medical literature, the accuracy of analysis is improved. Some or all of the above-described processing in the analysis unit may be performed using AI or without using AI. For example, the analysis unit can input medical literature data into the generative AI and cause the generative AI to improve the accuracy of analysis. Specifically, the analysis unit inputs paper texts obtained from PubMed and domestic and international medical literature databases (e.g., abstracts, full texts, figure captions), metadata (e.g., publication year, author, keywords), and related case image data into a natural language processing AI or image analysis AI. Examples of AI input include (1) abstracts of papers published in 2023 on infant growth abnormalities, (2) case images of dental malalignment and diagnostic criteria text, and (3) full text of the latest diagnostic guidelines. The analysis unit inputs these data into a Transformer-based large language model or multimodal AI and automatically extracts useful knowledge, rules, thresholds, and features for growth data analysis. The AI generates output such as a list of diagnostic criteria extracted from papers (e.g., “threshold for abnormal height-weight ratio in two-year-olds”), algorithm optimization parameters (e.g., “set threshold to 2.0 based on new guidelines”), and explanatory text for literature reference (e.g., “Abnormality judgment criteria updated in the latest paper”). The analysis unit incorporates these outputs into the analysis pipeline and also utilizes them for explanatory support of analysis results and strengthening accountability in medical settings. As a technical effect, the analysis unit realizes automatic reflection of the latest findings, improvement of analysis accuracy, and enhancement of explainability by AI, which was difficult with manual literature research or static rule updates by humans, thereby greatly improving reliability, reproducibility, and standardization in medical settings. Specific application fields include evidence-linked abnormality detection for infant checkups, automatic guideline updates for health management systems, and enhanced explanatory support for diagnostic AI in medical institutions.

The creation unit is configured to estimate the parent's emotion and adjust the content of the referral letter based on the estimated emotion. For example, if the parent is nervous, the creation unit creates a simple and easy-to-understand referral letter. If the parent is relaxed, the creation unit can create a referral letter including detailed information. Furthermore, if the parent is in a hurry, the creation unit can create a referral letter that focuses on key points. By adjusting the content of the referral letter according to the parent's emotion, the parent's understanding is deepened. Emotion estimation is realized using, for example, an emotion engine or generative AI with emotion estimation functions. The generative AI may be a text generative AI (e.g., LLM) or a multimodal generative AI, but is not limited to these examples. Some or all of the above-described processing in the creation unit may be performed using AI or without using AI. For example, the creation unit can input the parent's emotion data into the generative AI and cause the generative AI to perform emotion estimation. Specifically, the creation unit inputs text data entered by the parent (e.g., natural language sentences such as “The content of the referral letter is difficult”, “Details are not necessary”), voice data (e.g., audio waveform data, mel spectrogram), and facial image data (e.g., facial expression image tensor, shape: 224×224×3) into the emotion estimation AI. The creation unit normalizes and extracts features from these data at the preprocessing unit and inputs them into a multimodal emotion estimation AI integrating CNN and Transformer. Examples of AI input include (1) text sequences such as “Concise referral letter” (up to 512 tokens), (2) spectrogram of the parent's speech, and (3) facial expression image tensor. The creation unit receives output from the AI such as emotion label (e.g., “nervous”, “relaxed”, “in a hurry”), emotion score (continuous value from 0.0 to 1.0), and explanatory text for the estimation (e.g., “Judged as nervous because the word ‘difficult’ is included in the input sentence”). Based on these outputs, the creation unit instructs the referral letter generation module to automatically select the referral letter template and items to be included, such as emphasizing only important items in large font and omitting detailed information for the “nervous” label, adding detailed test results, information for the physician, and explanations of abnormality detection for the “relaxed” label, and listing only key points in bullet points and shortening the overall text for the “in a hurry” label. The creation unit reconstructs the content of the referral letter in real time when the parent's emotional state changes, always maintaining optimal information presentation. Examples of AI input include (1) text such as “Make the referral letter concise”, (2) spectrogram of the parent's speech, and (3) facial expression image tensor. Examples of AI output include (1) emotion label “nervous”, (2) emotion score 0.85, and (3) explanation “The word ‘difficult’ is included in the input sentence”. Based on these outputs, the creation unit automates selection of referral letter templates and inclusion or exclusion of items, generating referral letters optimized for the parent's psychological state. As a technical effect, the creation unit achieves high-precision emotion estimation and dynamic optimization of referral letters by AI, without relying on human subjective judgment or static referral letter template design, thereby improving understanding of referral letter content, preventing confusion due to information overload, and improving user satisfaction through user-specific optimization. The system can flexibly respond to user-specific information needs, which was difficult with conventional uniform referral letters, and also contributes to support for informed consent and strengthening accountability in medical settings. Specific application fields include emotion-adaptive referral letter generation for infant checkup apps, stress-responsive document creation for home health management systems, and personalized automatic generation of referral letters for medical institutions.

The creation unit is configured to generate appropriate content by referring to past referral letter data when creating the referral letter. For example, the creation unit generates optimal content based on past referral letter data. The creation unit can also analyze past referral letter data and optimize the content of the referral letter. Furthermore, the creation unit can refer to past referral letter data to improve the accuracy of the referral letter. By referring to past referral letter data, the accuracy of the referral letter content is improved. Some or all of the above-described processing in the creation unit may be performed using AI or without using AI. For example, the creation unit can input past referral letter data into the generative AI and cause the generative AI to generate appropriate content. Specifically, the creation unit inputs a database of past referral letter data (e.g., PDF files, text data, referral letter templates, referral letters with feedback from physicians) as structured data (e.g., text sequences for each referral letter item, abnormality content labels, medical department information, referral destination medical institution ID, etc.) into the AI. Examples of AI input include (1) referral letter text for the past year (up to 2048 tokens), (2) correspondence table of abnormality content and medical department, and (3) referral letter data with feedback from physicians. The creation unit inputs these data into a Transformer-based large language model or similarity search AI and searches and extracts the past cases most similar to the input growth data and abnormality content. The AI generates output such as the optimal referral letter template, list of items to be included (e.g., “dental alignment image”, “abnormality score”, “recommended department: pediatric dentistry”), and explanatory text for content optimization (e.g., “This description was effective in past similar cases”). Examples of AI output include (1) referral letter template “for infant dental abnormality”, (2) items to be included “dental alignment image”, “abnormality score”, “recommended department: pediatric dentistry”, and (3) reason “Received high evaluation from physicians in past similar cases”. Based on these outputs, the creation unit automatically generates the content of the referral letter and creates highly accurate referral letters reflecting effectiveness and feedback from past medical practice. Furthermore, after creating the referral letter, the creation unit continuously collects feedback from physicians and reinvests it as training data for the AI model, thereby achieving continuous optimization and improvement of referral letter content. As a technical effect, the creation unit realizes automatic generation of referral letters with high optimization, reproducibility, and explainability based on past cases by AI, which was difficult with manual case search or static template design by humans, thereby greatly improving reliability, standardization, and operational efficiency in medical practice. Specific application fields include automatic generation of referral letters for infant checkups, optimization of referral letter templates for medical institutions, and construction of referral letter databases for promoting medical DX.

The creation unit can customize the format of the referral letter based on the child's growth data when creating the referral letter. For example, the creation unit selects the optimal format based on the child's growth data. Additionally, the creation unit can analyze the child's growth data and customize the format of the referral letter. Furthermore, the creation unit can adjust the format of the referral letter according to the child's specific health condition. By customizing the format of the referral letter based on the child's growth data, the accuracy of the referral letter content is improved. Some or all of the above-described processing in the creation unit may be performed using AI, or may be performed without using AI. For example, the creation unit can input the child's growth data into a generative AI and have the generative AI execute the format customization. Specifically, the creation unit inputs the child's growth data (e.g., height, weight, dental alignment images, walking behavior, health status labels, etc.) as structured vectors (e.g., numerical vectors, image tensors, text sequences) into the AI. Examples of input to the AI include: (1) numerical vectors for height, weight, age, and gender (e.g., [90, 13, 2, 0]); (2) dental alignment image tensor (shape: 224×224×3); and (3) categorical data such as health status “allergy present”. The creation unit inputs these data into a multimodal AI (CNN+Transformer), which automatically selects the optimal referral letter format (e.g., item order, presence/absence of items, presence/absence of image attachments, department-specific templates, etc.) according to the content of the growth data, abnormal areas, and health status. The AI generates as output the format specifications (e.g., “template for dental alignment abnormality”, “image attachment required”, “department: pediatric dentistry”), a list of items to be described (e.g., “height”, “weight”, “abnormality details”, “image”), and a text explaining the reason for customization (e.g., “Image attachment required because dental alignment abnormality was detected”). Examples of AI output include: (1) format “for dental alignment abnormality”; (2) items “image attachment”, “abnormality score”; and (3) reason “health status includes malocclusion”. Based on these outputs, the creation unit automatically generates the layout and content of the referral letter and creates a referral letter optimized for each child. Furthermore, the creation unit can reconfigure the format in real time when the health status or growth data changes, always providing referral letters based on the latest medical information. As a technical effect, the creation unit automates dynamic format optimization according to the content of growth data using AI, which is difficult with manual format design or static template operation, thereby greatly improving the accuracy, explainability, and utility of referral letter content in medical settings. Specific application fields include individualized referral letter generation for infant health checkups, automatic generation of referral letter templates by health status, and automated personalization of referral letters for medical institutions.

The creation unit can estimate the parent's emotion and determine the priority of referral letters based on the estimated emotion. For example, if the parent is feeling stressed, the creation unit prioritizes the creation of the most important referral letters. If the parent is relaxed, the creation unit can sequentially create detailed referral letters. Furthermore, if the parent is in a hurry, the creation unit can prioritize the creation of referral letters that focus on key points. By determining the priority of referral letters according to the parent's emotion, important referral letters can be created preferentially. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the creation unit may be performed using AI, or may be performed without using AI. For example, the creation unit can input the parent's emotion data into a generative AI and have the generative AI execute emotion estimation. Specifically, the creation unit inputs text data entered by the parent (e.g., natural language sentences such as “I'm in a hurry” or “Only the important content is needed”), audio data (e.g., audio waveforms, mel spectrograms), and facial image data (e.g., facial expression image tensors) into an emotion estimation AI. Examples of input to the AI include: (1) text sequences such as “I'm in a hurry” (up to 512 tokens); (2) spectrograms of the parent's speech (shape: 128×256); and (3) facial expression image tensors (shape: 224×224×3). The creation unit inputs these data into a CNN+Transformer integrated model, which classifies emotion categories (e.g., stress, relaxation, hurry). The AI generates as output emotion labels (e.g., “in a hurry”), emotion scores (0.0 to 1.0), and text explaining the basis for estimation (e.g., “The input sentence contains ‘in a hurry’”). Based on these outputs, the creation unit determines the priority for creating referral letters, for example, creating “cases with high abnormality” or “items requiring attention” first, and postponing detailed explanations or supplementary information. Furthermore, when the parent's emotional state changes, the creation unit reconfigures the creation order in real time, always maintaining optimal referral letter creation. As a technical effect, the creation unit achieves high-precision emotion estimation and dynamic priority control by AI, without relying on human subjective judgment or static creation order design, thereby preventing the omission of important information, improving user satisfaction, and streamlining referral letter creation operations. Specific application fields include emotion-adaptive referral letter creation for infant health checkup apps, stress-responsive document prioritization in home health management systems, and personalized creation order control for referral letters for medical institutions.

The creation unit can select the optimal hospital by considering the child's geographic location information when creating the referral letter. For example, the creation unit selects the nearest hospital based on the child's current location. Additionally, the creation unit can propose the optimal hospital based on the child's geographic location information. Furthermore, the creation unit can select the optimal hospital by considering the child's movement range. By selecting the optimal hospital based on the child's geographic location information, the child can receive prompt and appropriate medical care. Some or all of the above-described processing in the creation unit may be performed using AI, or may be performed without using AI. For example, the creation unit can input the child's geographic location information data into a generative AI and have the generative AI execute the selection of the optimal hospital. Specifically, the creation unit normalizes GPS coordinate data obtained from the child's device (e.g., numerical vectors for latitude and longitude, e.g., 35.6895, 139.6917), region codes linked to location information (e.g., prefecture, city/ward/town/village ID), and movement history (e.g., time-series vectors of location information for the past week, shape: 7×2) in a preprocessing unit and inputs them into the AI. Examples of input to the AI include: (1) “Shinjuku-ku, Tokyo, current location: 35.6938, 139.7034”; (2) “Travel destination: Kita-ku, Osaka, movement history: Tokyo→Osaka”; and (3) “Planned relocation: Nishi-ku, Yokohama” as structured data. The creation unit inputs these geographic information into a Transformer-based location information analysis AI, which refers to region-specific medical institution lists (e.g., departments, consultation hours, specialties, congestion status, etc.) and infers optimal hospital candidates. The AI calculates relevance scores from the input geographic information and generates as output a list of nearby hospitals (e.g., “Shinjuku Pediatric Clinic”, “Kita-ku Children's Hospital, Osaka”), priority for each hospital (e.g., distance score 0.95, specialty score 0.90), and text explaining the reason for selection (e.g., “5 minutes walk from current location, specialist available”). Examples of AI output include: (1) hospital candidate “Shinjuku Pediatric Clinic”; (2) priority “distance 0.95, specialty 0.90”; and (3) reason “shortest from current location”. Based on these outputs, the creation unit automatically records the optimal hospital information in the referral letter, supporting the parent in receiving prompt and appropriate medical care. Furthermore, when the parent or child's movement plans change, the creation unit immediately reconfigures hospital candidates and always provides the latest medical institution information. As a technical effect, the creation unit automates dynamic hospital optimization based on geographic information using AI, which is difficult with manual hospital search or static hospital list operation, thereby improving the convenience of referral letters, speeding up medical consultations, and enhancing medical access. Specific application fields include region-adaptive referral letter generation for infant health checkups, automatic proposal of nearby medical institutions in home health management systems, and region-specific automation of referral letters for medical institutions.

The creation unit can refer to relevant medical literature when creating the referral letter to enrich its content. For example, the creation unit refers to the latest medical literature to enhance the content of the referral letter. Additionally, the creation unit can improve the accuracy of the referral letter based on relevant medical literature. Furthermore, the creation unit can incorporate medical literature data into the referral letter to provide more accurate content. By referring to relevant medical literature, the accuracy of the referral letter content is improved. Some or all of the above-described processing in the creation unit may be performed using AI, or may be performed without using AI. For example, the creation unit can input medical literature data into a generative AI and have the generative AI execute the enrichment of the referral letter content. Specifically, the creation unit inputs paper texts obtained from PubMed or domestic and international medical journal databases (e.g., abstracts, main texts, figure/table captions), metadata (e.g., publication year, authors, keywords), and relevant case image data into a natural language processing AI or image analysis AI. Examples of input to the AI include: (1) abstracts of papers on infant growth abnormalities published in 2023; (2) case images of malocclusion and diagnostic criteria texts; and (3) full text of the latest diagnostic guidelines. The creation unit inputs these data into a Transformer-based large language model or multimodal AI, which automatically extracts useful diagnostic criteria, treatment policies, latest findings, and evidence for referral letter content. The AI generates as output a list of diagnostic criteria extracted from papers (e.g., “threshold for abnormal height-to-weight ratio in 2-year-olds”), evidence text for referral letter description (e.g., “abnormality judgment criteria updated based on latest paper”), and text explaining the reason for literature reference (e.g., “compliant with guidelines published in 2023”). Examples of AI output include: (1) diagnostic criteria “height-to-weight ratio less than 2.0 is judged as abnormal”; (2) evidence text “abnormality judgment criteria updated in latest paper”; and (3) reason “compliant with guidelines published in 2023”. Based on these outputs, the creation unit automatically incorporates the latest medical findings and evidence into the referral letter content, strengthening accountability and diagnostic accuracy in medical settings. Furthermore, the creation unit automatically updates the referral letter content according to updates in the medical literature database, always providing referral letters reflecting the latest medical information. As a technical effect, the creation unit realizes automatic reflection of the latest findings, improvement of referral letter content accuracy, and enhancement of explainability by AI, which were difficult with manual literature surveys or static rule updates, thereby greatly improving reliability, reproducibility, and standardization in medical settings. Specific application fields include evidence-linked referral letter generation for infant health checkups, automatic guideline updates in health management systems, and enhancement of rationale explanation for referral letters for medical institutions.

The distribution unit can estimate the parent's emotion and adjust the distribution method of the referral letter based on the estimated emotion. For example, if the parent is nervous, the distribution unit provides a simple and easy-to-understand distribution method. If the parent is relaxed, the distribution unit can provide a distribution method that includes detailed information. Furthermore, if the parent is in a hurry, the distribution unit can provide a method that enables rapid distribution. By adjusting the distribution method of the referral letter according to the parent's emotion, the burden on the parent is reduced. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the distribution unit may be performed using AI, or may be performed without using AI. For example, the distribution unit can input the parent's emotion data into a generative AI and have the generative AI execute emotion estimation. Specifically, the distribution unit inputs text data entered by the parent (e.g., natural language sentences such as “I'm in a hurry” or “The distribution method is difficult to understand”), audio data (e.g., audio waveform data, mel spectrograms), and facial image data (e.g., facial expression image tensors, shape: 224×224×3) into an emotion estimation AI. Examples of input to the AI include: (1) text sequences such as “The distribution method is difficult” (integer array up to 512 tokens); (2) mel spectrograms of the parent's speech (shape: 128×256); and (3) facial expression image tensors (shape: 224×224×3). The distribution unit normalizes and extracts features from these input data in a preprocessing unit and inputs them into a multimodal emotion estimation AI integrating CNN and Transformer. The AI classifies emotion categories (e.g., nervousness, relaxation, hurry) from the input data and generates as output emotion labels (e.g., “nervous”), emotion scores (continuous values from 0.0 to 1.0), and text explaining the basis for emotion estimation (e.g., “Judged as nervous because the input sentence contains ‘difficult’”). Examples of AI output include: (1) emotion label “nervous”; (2) emotion score 0.85; and (3) explanation “Facial expression is tense and the input sentence contains ‘difficult’”. Based on these outputs, the distribution unit instructs the distribution method generation module, for example, to simplify the email body and provide illustrated instructions for the “nervous” label, to present detailed options (e.g., email, app notification, cloud storage, mail, etc.) and add explanations of distribution history and download methods for the “relaxed” label, and to place a one-click download link at the top and send distribution notifications immediately for the “in a hurry” label. Furthermore, when the parent's emotional state changes, the distribution unit reconfigures the distribution method in real time, maintaining the optimal distribution experience even during input. As a technical effect, the distribution unit achieves high-precision emotion estimation and dynamic optimization of distribution methods by AI, without relying on human subjective judgment or static distribution method design, thereby greatly reducing psychological burden, distribution errors, and receipt delays. The system automates user-specific optimization, which was difficult with conventional uniform distribution methods, and improves overall system usability and distribution reliability. Specific application fields include emotion-adaptive referral letter distribution for infant health checkup apps, stress-responsive distribution support in home health management systems, and automated personalized distribution of referral letters for medical institutions.

The distribution unit can refer to the parent's past distribution history when distributing referral letters to select the optimal distribution method. For example, the distribution unit selects the optimal distribution method based on the distribution methods previously used by the parent. Additionally, the distribution unit can analyze the parent's past distribution history to optimize the distribution method. Furthermore, the distribution unit can refer to the parent's past distribution history to improve the accuracy of distribution. By referring to the parent's past distribution history, the optimal distribution method can be selected. Some or all of the above-described processing in the distribution unit may be performed using AI, or may be performed without using AI. For example, the distribution unit can input the parent's past distribution history data into a generative AI and have the generative AI execute the selection of the optimal distribution method. Specifically, the distribution unit inputs a distribution history database accumulated for each parent (e.g., structured data such as distribution date and time for the past year, distribution method, recipient address, success/failure flag, receipt confirmation date and time, device type, etc.) as time-series vectors (e.g., vectorizing each history entry, shape: N×M, where N is the number of histories and M is the number of features) into the AI. Examples of input to the AI include: (1) history sequences such as “2023/05/01 20:00, email, success, smartphone”, “2023/06/01 21:00, app notification, failure, tablet”; (2) success rate statistics by distribution method (e.g., email 90%, app notification 80%); and (3) category distribution of recipient devices (e.g., smartphone 70%, PC 30%). The distribution unit inputs these history data into an LSTM or Transformer-based time-series analysis AI, which extracts distribution patterns for each parent. The AI predicts the distribution method and recipient with the highest success rate for the next distribution based on the distribution history and generates as output a recommended distribution method label (e.g., “email distribution recommended”), a list of candidate recipients (e.g., “smartphone”, “cloud storage”), and text explaining the recommendation (e.g., “Email distribution succeeded three times in a row”). Examples of AI output include: (1) recommended distribution method “email”; (2) candidate recipients “smartphone”, “cloud storage”; and (3) recommendation reason “Email receipt rate is high at night”. Based on these outputs, the distribution unit automatically generates the distribution interface and instantly presents the optimal distribution method and recipient to the parent when receiving the referral letter. Furthermore, the distribution unit monitors the parent's distribution receipt behavior in real time and dynamically updates the distribution method and candidate recipients. As a technical effect, the distribution unit automates personalized distribution support based on user-specific distribution tendencies using AI, which is difficult with manual history analysis or static candidate presentation, thereby improving distribution efficiency, reducing distribution errors, and enhancing user satisfaction. Specific application fields include history-learning-based distribution support for infant health checkup apps, personalized distribution forms for home health management systems, and automatic candidate presentation for referral letter distribution for medical institutions.

The distribution unit can customize the distribution means based on the parent's current living situation when distributing referral letters. For example, if the parent is busy, the distribution unit distributes the referral letter quickly via email. If the parent is at home, the distribution unit can distribute the referral letter by mail. Furthermore, if the parent is traveling, the distribution unit can distribute the referral letter in digital format. By customizing the distribution means according to the parent's living situation, distribution efficiency is improved. Some or all of the above-described processing in the distribution unit may be performed using AI, or may be performed without using AI. For example, the distribution unit can input the parent's living situation data into a generative AI and have the generative AI execute customization of the distribution means. Specifically, the distribution unit inputs living situation data entered by the parent (e.g., occupation, working hours, at-home/outdoor status, travel plans, device usage status, communication environment, etc. as text/numerical data) as structured vectors (e.g., one-hot encoding by category, normalized numerical values) into the AI. Examples of input to the AI include: (1) “Occupation: company employee, working hours: 9-18, at home: 2 days a week, device: smartphone”; (2) “Traveling, communication environment: unstable, device: tablet”; and (3) “Staying at home, mail receipt available”. The distribution unit inputs these data into a Transformer-based feature extraction AI, which infers the optimal set of distribution means for the parent's living situation. The AI calculates importance scores from the input data and generates as output a list of distribution means (e.g., “email”, “app notification”, “mail”), priority for each means (e.g., importance score 0.95), and text explaining the reason for customization (e.g., “Digital distribution prioritized due to travel”). Examples of AI output include: (1) distribution means “email”, “app notification”; (2) priority “email: 0.95, app notification: 0.90”; and (3) reason “Email is easily checked during working hours”. Based on these outputs, the distribution unit automatically generates the distribution means and adds, deletes, or reorders means in real time according to the parent's situation. Furthermore, when the parent's living situation changes, the distribution unit immediately reconfigures the distribution means, always providing the optimal distribution experience. As a technical effect, the distribution unit automates dynamic optimization of distribution means according to each user's situation using AI, which is difficult with manual customization or static distribution method design, thereby improving distribution efficiency, standardizing distribution quality, and enhancing user satisfaction. Specific application fields include lifestyle-adaptive distribution support for infant health checkup apps, automatic selection of distribution means by situation in home health management systems, and automated personalized distribution of referral letters for medical institutions.

The distribution unit can estimate the parent's emotion and determine the priority of referral letter distribution based on the estimated emotion. For example, if the parent is feeling stressed, the distribution unit prioritizes the distribution of the most important referral letters. If the parent is relaxed, the distribution unit can sequentially distribute detailed referral letters. Furthermore, if the parent is in a hurry, the distribution unit can prioritize the distribution of referral letters that focus on key points. By determining the priority of referral letter distribution according to the parent's emotion, important referral letters can be distributed preferentially. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the distribution unit may be performed using AI, or may be performed without using AI. For example, the distribution unit can input the parent's emotion data into a generative AI and have the generative AI execute emotion estimation. Specifically, the distribution unit inputs text data entered by the parent (e.g., natural language sentences such as “I'm in a hurry” or “Only the important referral letters are needed”), audio data (e.g., audio waveforms, mel spectrograms), and facial image data (e.g., facial expression image tensors) into an emotion estimation AI. Examples of input to the AI include: (1) text sequences such as “I'm in a hurry” (up to 512 tokens); (2) spectrograms of the parent's speech (shape: 128×256); and (3) facial expression image tensors (shape: 224×224×3). The distribution unit inputs these data into a CNN+Transformer integrated model, which classifies emotion categories (e.g., stress, relaxation, hurry). The AI generates as output emotion labels (e.g., “in a hurry”), emotion scores (0.0 to 1.0), and text explaining the basis for estimation (e.g., “The input sentence contains ‘in a hurry’”). Based on these outputs, the distribution unit instructs the distribution priority determination module, for example, to distribute referral letters with high abnormality or items requiring attention first for the “stress” label, to sequentially distribute detailed explanations and supplementary information for the “relaxation” label, and to preferentially distribute referral letters listing only key points for the “in a hurry” label. Furthermore, when the parent's emotional state changes, the distribution unit reconfigures the distribution order in real time, always maintaining the optimal distribution experience. As a technical effect, the distribution unit achieves high-precision emotion estimation and dynamic priority control by AI, without relying on human subjective judgment or static distribution order design, thereby preventing the omission of important information, improving user satisfaction, and streamlining distribution operations. Specific application fields include emotion-adaptive referral letter distribution for infant health checkup apps, stress-responsive distribution prioritization in home health management systems, and personalized distribution order control for referral letters for medical institutions.

The distribution unit can select the optimal distribution means by considering the parent's geographic location information when distributing referral letters. For example, if the parent lives in a specific region, the distribution unit selects the distribution means optimal for that region. Additionally, if the parent is traveling, the distribution unit can select the distribution means optimal for the travel destination. Furthermore, if the parent is planning to move, the distribution unit can select the distribution means optimal for the new region. By selecting the optimal distribution means based on the parent's geographic location information, rapid and appropriate distribution is possible. Some or all of the above-described processing in the distribution unit may be performed using AI, or may be performed without using AI. For example, the distribution unit can input the parent's geographic location information data into a generative AI and have the generative AI execute the selection of the optimal distribution means. Specifically, the distribution unit normalizes GPS coordinate data obtained from the parent's device (e.g., numerical vectors for latitude and longitude, e.g., 35.6895, 139.6917), region codes linked to location information (e.g., prefecture, city/ward/town/village ID), and movement history (e.g., time-series vectors of location information for the past week, shape: 7×2) in a preprocessing unit and inputs them into the AI. Examples of input to the AI include: (1) “Shinjuku-ku, Tokyo, current location: 35.6938, 139.7034”; (2) “Travel destination: Kita-ku, Osaka, movement history: Tokyo→Osaka”; and (3) “Planned relocation: Nishi-ku, Yokohama” as structured data. The distribution unit inputs these geographic information into a Transformer-based location information analysis AI, which refers to region-specific distribution means (e.g., mail availability, electronic distribution availability, region-limited app notifications, etc.) and infers optimal distribution means candidates. The AI calculates relevance scores from the input geographic information and generates as output a list of optimal distribution means (e.g., “email”, “mail”, “app notification”), priority for each means (e.g., distance score 0.92, availability score 0.88), and text explaining the reason for selection (e.g., “Mail is rapid in this region”). Examples of AI output include: (1) distribution means “email”, “mail”; (2) priority “email: 0.92, mail: 0.88”; and (3) reason “Electronic distribution is optimal at travel destination”. Based on these outputs, the distribution unit automatically generates the distribution means and adds, deletes, or reorders means in real time according to the parent's current location or movement plans. Furthermore, when the parent's location information changes, the distribution unit immediately reconfigures the distribution means, always providing the optimal distribution experience. As a technical effect, the distribution unit automates dynamic optimization of distribution means according to the user's geographic situation using AI, which is difficult with manual customization or static distribution method design, thereby improving distribution efficiency, standardizing distribution quality, and enhancing user satisfaction. The system can flexibly respond to region-specific communication infrastructure and mail circumstances that could not be addressed by conventional uniform distribution means, thereby improving the accuracy of referral letter receipt and greatly enhancing the reliability of subsequent medical access. Specific application fields include region-adaptive distribution support for infant health checkup apps, automatic selection of distribution means by region in home health management systems, and region-specific automated distribution of referral letters for medical institutions.

The distribution unit can analyze the parent's social media activity when distributing referral letters to propose distribution means. For example, the distribution unit proposes optimal distribution means based on information shared by the parent on social media. Additionally, the distribution unit can propose optimal distribution means based on accounts followed by the parent on social media. Furthermore, the distribution unit can propose optimal distribution means based on information about groups the parent participates in on social media. By analyzing the parent's social media activity, optimal distribution means can be proposed. Some or all of the above-described processing in the distribution unit may be performed using AI, or may be performed without using AI. For example, the distribution unit can input the parent's social media activity data into a generative AI and have the generative AI execute the proposal of distribution means. Specifically, the distribution unit stores post data obtained from multiple social media platforms used by the parent (e.g., text posts, image posts, video links), lists of followed accounts (e.g., arrays of health/childcare-related account IDs), and group participation information (e.g., childcare group names, activity frequency, summaries of post content) in a structured database, performs natural language processing, image analysis, and category classification in a preprocessing unit, and inputs the data into the AI. Examples of input to the AI include: (1) “2023/06/01 post: wishes to share referral letter on SNS”; (2) “Followed accounts: official medical institution, health management app” as text/image data; and (3) “Participating group: Infant Health Management Association, posts once a week” as group activity data. The distribution unit inputs these data into a Transformer-based multimodal AI, which infers highly relevant distribution means (e.g., SNS-linked distribution, direct message distribution, cloud storage sharing, etc.) from the parent's areas of interest, recent topics, and post content. The AI extracts features from the input data and generates as output a list of proposed distribution means (e.g., “SNS linkage”, “cloud storage sharing”, “email”), priority for each means (e.g., importance score 0.88), and text explaining the reason for the proposal (e.g., “Recent post indicates desire for SNS sharing”). Examples of AI output include: (1) distribution means “SNS linkage”, “cloud storage sharing”; (2) priority “SNS linkage: 0.88, cloud storage sharing: 0.82”; and (3) reason “Followed account is a health management app”. Based on these outputs, the distribution unit automatically generates the distribution means and adds, deletes, or reorders means in real time according to the parent's social media activity. Furthermore, when the parent's new posts or group participation status changes, the distribution unit immediately reconfigures the distribution means, always providing the optimal distribution experience. As a technical effect, the distribution unit automates dynamic optimization of distribution means according to the user's online activity using AI, which is difficult with manual customization or static distribution method design, thereby improving distribution efficiency, standardizing distribution quality, and enhancing user satisfaction. The system can flexibly respond to individual interests and the latest topics that could not be addressed by conventional uniform distribution means, thereby improving the accuracy of referral letter receipt and greatly enhancing the reliability of subsequent medical access. Specific application fields include SNS-linked distribution support for infant health checkup apps, automatic extraction of areas of interest for distribution in home health management systems, and automated online activity-linked distribution of referral letters for medical institutions.

The learning unit can estimate the parent's emotion and select learning data based on the estimated emotion. For example, if the parent is feeling stressed, the learning unit selects simple and intuitive learning data. If the parent is relaxed, the learning unit can select detailed learning data. Furthermore, if the parent is in a hurry, the learning unit can select learning data that focuses on key points. By selecting learning data according to the parent's emotion, learning efficiency is improved. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the learning unit may be performed using AI, or may be performed without using AI. For example, the learning unit can input the parent's emotion data into a generative AI and have the generative AI execute emotion estimation. Specifically, the learning unit inputs text data entered by the parent (e.g., natural language sentences such as “I'm tired today” or “I want the learning content to be simple”), audio data (e.g., audio waveform data, mel spectrograms), and facial image data (e.g., facial expression image tensors, shape: 224×224×3) into an emotion estimation AI. Examples of input to the AI include: (1) text sequences such as “Make the learning content simple” (integer array up to 512 tokens); (2) mel spectrograms of the parent's speech (shape: 128×256); and (3) facial expression image tensors (shape: 224×224×3). The learning unit normalizes and extracts features from these input data in a preprocessing unit and inputs them into a multimodal emotion estimation AI integrating CNN and Transformer. The AI classifies emotion categories (e.g., stress, relaxation, hurry) from the input data and generates as output emotion labels (e.g., “stress”), emotion scores (continuous values from 0.0 to 1.0), and text explaining the basis for emotion estimation (e.g., “Judged as stress because the input sentence contains ‘tired’”). Examples of AI output include: (1) emotion label “stress”; (2) emotion score 0.81; and (3) explanation “Facial expression is tense and the input sentence contains ‘tired’”. Based on these outputs, the learning unit instructs the learning data selection module, for example, to automatically select basic content and illustration-focused materials for the “stress” label, materials with detailed explanations and applied problems for the “relaxation” label, and summary materials focusing only on key points for the “in a hurry” label. Furthermore, when the parent's emotional state changes, the learning unit reconfigures the content and difficulty of the learning data in real time, always providing the optimal learning experience. Examples of input to the AI include: (1) text such as “Only the key points are needed for learning”; (2) spectrograms of the parent's speech; and (3) facial expression image tensors. Examples of AI output include: (1) emotion label “in a hurry”; (2) emotion score 0.92; and (3) explanation “The input sentence contains ‘key points’”. Based on these outputs, the learning unit automates the selection of learning data templates and content, providing learning data optimized for the parent's psychological state. As a technical effect, the learning unit achieves high-precision emotion estimation and dynamic optimization of learning data by AI, without relying on human subjective judgment or static learning data design, thereby improving understanding of learning content, preventing confusion due to information overload, and enhancing satisfaction through user-specific optimization. The system enables flexible response to individual information needs, which was difficult with conventional uniform learning data, and contributes to personalized learning support and strengthened accountability in educational settings. Specific application fields include emotion-adaptive learning material generation for infant health checkup apps, stress-responsive learning support in home health management systems, and automated personalized generation of learning materials for medical institutions.

The learning unit can optimize the learning algorithm by referring to past learning data during learning. For example, the learning unit selects the optimal learning algorithm based on past learning data. Additionally, the learning unit can analyze past learning data and adjust the parameters of the learning algorithm. Furthermore, the learning unit can improve the accuracy of the learning algorithm by referring to past learning data. By referring to past learning data, the accuracy of the learning algorithm is improved. Some or all of the above-described processing in the learning unit may be performed using AI, or may be performed without using AI. For example, the learning unit can input past learning data into a generative AI and have the generative AI execute optimization of the learning algorithm. Specifically, the learning unit inputs a past learning database (e.g., learning history, correct answer rate trends, error patterns, types of learning materials, learning time, learning frequency, etc. as structured data) as time-series vectors (shape: N×M, where N is the number of data entries and M is the number of features), text sequences (up to 512 tokens), and image data (learning material images and explanatory diagrams, shape: 224×224×3) into the AI. Examples of input to the AI include: (1) learning history vectors for the past year (e.g., date, material ID, correct answer rate, learning time); (2) text records of error patterns; and (3) learning material image data. The learning unit inputs these data into the AI using supervised learning, transfer learning, or self-supervised learning methods, and the AI automatically selects the optimal algorithm from multiple algorithms such as CNN, Transformer, decision tree, and random forest. The AI performs cross-validation and grid search on the input data and generates as output the name of the optimal algorithm (e.g., “LSTM+Attention”), parameter set (e.g., “learning rate=0.001, batch size=32”), and accuracy evaluation score (e.g., AUC=0.94, F1=0.91). Examples of AI output include: (1) optimal algorithm “LSTM+Attention”; (2) parameters “learning rate 0.001, batch size 32”; and (3) accuracy evaluation “AUC=0.94”. Based on these outputs, the learning unit automatically configures the learning pipeline, strengthening reproducibility, generalizability, and the ability to discover new error patterns in learning results. Furthermore, the learning unit continuously readjusts parameters based on feedback from learning results to improve model accuracy. As a technical effect, the learning unit realizes automatic design and optimization of the optimal AI learning pipeline according to data characteristics and learning objectives, which is difficult with manual algorithm selection or static rule-based processing, thereby greatly improving learning accuracy, reproducibility, and generalizability. This enables individualized learning support, early detection of error patterns, and standardization of learning methods in educational settings. Specific application fields include automatic learning optimization for infant health checkup apps, educational big data analysis platforms, and personalized optimization of learning materials for medical institutions.

The learning unit can estimate the parent's emotion and adjust the frequency of learning based on the estimated emotion. For example, if the parent is feeling stressed, the learning unit reduces the frequency of learning. If the parent is relaxed, the learning unit can increase the frequency of learning. Furthermore, if the parent is in a hurry, the learning unit can adjust the frequency of learning. By adjusting the frequency of learning according to the parent's emotion, learning efficiency is improved. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the learning unit may be performed using AI, or may be performed without using AI. For example, the learning unit can input the parent's emotion data into a generative AI and have the generative AI execute emotion estimation. Specifically, the learning unit inputs text data entered by the parent (e.g., natural language sentences such as “I'm busy today” or “I want to keep learning to a minimum”), audio data (e.g., audio waveform data, mel spectrograms), and facial image data (e.g., facial expression image tensors, shape: 224×224×3) into an emotion estimation AI. Examples of input to the AI include: (1) text sequences such as “Keep learning to a minimum” (integer array up to 512 tokens); (2) mel spectrograms of the parent's speech (shape: 128×256); and (3) facial expression image tensors (shape: 224×224×3). The learning unit normalizes and extracts features from these input data in a preprocessing unit and inputs them into a multimodal emotion estimation AI integrating CNN and Transformer. The AI classifies emotion categories (e.g., stress, relaxation, hurry) from the input data and generates as output emotion labels (e.g., “stress”), emotion scores (continuous values from 0.0 to 1.0), and text explaining the basis for emotion estimation (e.g., “Judged as stress because the input sentence contains ‘minimum’”). Examples of AI output include: (1) emotion label “stress”; (2) emotion score 0.78; and (3) explanation “Facial expression is tense and the input sentence contains ‘minimum’”. Based on these outputs, the learning unit instructs the learning frequency control module, for example, to limit learning frequency to once a week for the “stress” label, recommend daily learning for the “relaxation” label, and divide short learning sessions into multiple times for the “in a hurry” label. Furthermore, when the parent's emotional state changes, the learning unit reconfigures the learning frequency in real time, always providing the optimal learning experience. Examples of input to the AI include: (1) text such as “I want to refrain from learning today”; (2) spectrograms of the parent's speech; and (3) facial expression image tensors. Examples of AI output include: (1) emotion label “stress”; (2) emotion score 0.80; and (3) explanation “The input sentence contains ‘refrain’”. Based on these outputs, the learning unit automatically adjusts the learning frequency and optimizes reminder notifications, realizing learning frequency optimized for the parent's psychological state. As a technical effect, the learning unit achieves high-precision emotion estimation and dynamic optimization of learning frequency by AI, without relying on human subjective judgment or static learning frequency design, thereby optimizing learning load, improving continuation rate, and enhancing user satisfaction. The system enables flexible adjustment of user-specific burden and continuation support, which was difficult with conventional uniform learning frequency, and contributes to personalized learning support and stress management in educational settings. Specific application fields include emotion-adaptive learning frequency control for infant health checkup apps, stress-responsive learning reminders in home health management systems, and personalized optimization of learning frequency for learning materials for medical institutions.

The learning unit can weight learning data based on the submission timing of growth data during learning. For example, the learning unit emphasizes recently submitted growth data in learning. Additionally, the learning unit can weight learning data based on growth data submitted during a specific period. Furthermore, the learning unit can adjust the importance of growth data according to the submission timing and conduct learning. By weighting learning data based on the submission timing of growth data, learning accuracy is improved. Some or all of the above-described processing in the learning unit may be performed using AI, or may be performed without using AI. For example, the learning unit can input submission timing data of growth data into a generative AI and have the generative AI execute weighting of learning data. Specifically, the learning unit vectorizes time-series data such as submission date and time of growth data (e.g., UNIX timestamp, YYYY/MM/DD format), submission interval, and submission frequency, and inputs them into the AI. Examples of input to the AI include: (1) history sequences such as “2023/05/01 20:00, height, 90 cm”, “2023/06/01 20:00, height, 91 cm”; (2) time-series arrays of submission intervals (e.g., 30 days, 31 days); and (3) statistical values of submission frequency (e.g., twice a month). The learning unit inputs these data into an LSTM or Transformer-based time-series analysis AI, which generates weighting vectors that assign higher weights to the latest data or data from specific periods. The AI generates as output a weighted growth data set, a list of weight values (e.g., latest data=1.0, one year ago=0.5), and text explaining the reason for weighting (e.g., “Weight increased due to high reliability of latest data”). Examples of AI output include: (1) weighted data set; (2) weight value list “latest=1.0, one year ago=0.5”; and (3) reason “Latest data is highly reliable”. Based on these outputs, the learning unit applies weight parameters to the learning algorithm, improving the accuracy of anomaly detection and growth assessment learning. Furthermore, when the submission timing changes or new data is added, the learning unit recalculates the weights in real time, always maintaining learning that reflects the latest information. As a technical effect, the learning unit automates dynamic optimization of time-series data weighting by AI, which was difficult with conventional uniform weighting or manual adjustment, thereby realizing reflection of the latest information, control of the influence of past data, and improvement of learning accuracy. Specific application fields include time-series growth learning for infant health checkups, latest data-focused learning in health management systems, and time-series anomaly detection learning for medical institutions.

The format unit can estimate the parent's emotion and adjust the design of the input format based on the estimated emotion. For example, if the parent is nervous, the format unit provides a format with calm colors to reduce visual stress. If the parent is enjoying the process, the format unit can provide a format with bright colors to make the input task enjoyable. Furthermore, if the parent is tired, the format unit can provide a simple and highly visible format to make input tasks easier. By adjusting the design of the input format according to the parent's emotion, input efficiency is improved. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the format unit may be performed using AI, or may be performed without using AI. For example, the format unit can input the parent's emotion data into a generative AI and have the generative AI execute emotion estimation. Specifically, the format unit inputs text data entered by the parent (e.g., natural language sentences such as “I'm tired today” or “Input task is difficult”), audio data (e.g., audio waveform data, mel spectrograms), and facial image data (e.g., facial expression image tensors, shape: 224×224×3) into an emotion estimation AI. Examples of input to the AI include: (1) text sequences such as “Input task is difficult” (integer array up to 512 tokens); (2) mel spectrograms of the parent's speech (shape: 128×256); and (3) facial expression image tensors (shape: 224×224×3). The format unit normalizes and extracts features from these input data in a preprocessing unit and inputs them into a multimodal emotion estimation AI integrating CNN and Transformer. The AI classifies emotion categories (e.g., nervousness, enjoyment, tiredness) from the input data and generates as output emotion labels (e.g., “tired”), emotion scores (continuous values from 0.0 to 1.0), and text explaining the basis for emotion estimation (e.g., “Judged as tired because the input sentence contains ‘difficult’”). Examples of AI output include: (1) emotion label “tired”; (2) emotion score 0.83; and (3) explanation “Facial expression is downcast and the input sentence contains ‘difficult’”. Based on these outputs, the format unit instructs the input format generation module, for example, to automatically generate a blue-toned calm color scheme and large font for the “nervous” label, a colorful color scheme and animation for the “enjoyment” label, and a simple layout and minimal input items for the “tired” label. Furthermore, when the parent's emotional state changes, the format unit reconfigures the format design in real time, always providing the optimal input experience. As a technical effect, the format unit achieves high-precision emotion estimation and dynamic optimization of the format by AI, without relying on human subjective judgment or static format design, thereby greatly reducing psychological burden, input errors, and dropout rates. The system automates user-specific optimization, which was difficult with conventional uniform formats, and improves overall system usability and data quality. Specific application fields include emotion-adaptive input format generation for infant health checkup apps, stress-responsive input support in home health management systems, and emotion-linked automatic generation of electronic medical questionnaires for medical institutions.

The format unit can refer to past input data when creating the input format to generate the optimal format. For example, the format unit generates the optimal format based on past input data. Additionally, the format unit can analyze past input data to optimize the format design. Furthermore, the format unit can improve the accuracy of the format by referring to past input data. By referring to past input data, the accuracy of the input format is improved. Some or all of the above-described processing in the format unit may be performed using AI, or may be performed without using AI. For example, the format unit can input past input data into a generative AI and have the generative AI execute generation of the optimal format. Specifically, the format unit inputs a past input database (e.g., input history, order of input items, input time, locations of input errors, input device information, etc. as structured data) as time-series vectors (shape: N×M, where N is the number of histories and M is the number of features), text sequences (up to 512 tokens), and input screen images (shape: 224×224×3) into the AI. Examples of input to the AI include: (1) input history vectors for the past year (e.g., date, item ID, input order, input time); (2) text records of input error locations; and (3) input screen image data. The format unit inputs these data into the AI using supervised learning, transfer learning, or self-supervised learning methods, and the AI automatically selects the optimal format from multiple algorithms such as CNN, Transformer, decision tree, and random forest. The AI performs cross-validation and grid search on the input data and generates as output the name of the optimal format (e.g., “simple type”, “detailed type”), parameter set (e.g., “item order: height→weight→dental alignment”), and accuracy evaluation score (e.g., input error rate 0.02, average input time 30 seconds). Examples of AI output include: (1) optimal format “simple type”; (2) parameters “item order: height→weight→dental alignment”; and (3) accuracy evaluation “input error rate 0.02”. Based on these outputs, the format unit automatically generates the input form, creating a highly accurate input format that reflects past input patterns and error tendencies. Furthermore, when input history changes or new data is added, the format unit reconfigures the format in real time, always providing the optimal input experience. As a technical effect, the format unit realizes automatic generation of highly optimized, reproducible, and explainable input formats based on past cases by AI, which is difficult with manual format design or static template operation, thereby greatly improving overall system reliability, standardization, and operational efficiency. Specific application fields include automatic optimization of input forms for infant health checkup apps, optimization of input templates for medical institutions, and construction of input databases for promoting educational DX.

The format unit can estimate the parent's emotion and determine the priority of input formats based on the estimated emotion. For example, if the parent is feeling stressed, the format unit preferentially displays the most important input formats. If the parent is relaxed, the format unit can sequentially display detailed input formats. Furthermore, if the parent is in a hurry, the format unit can preferentially display input formats that focus on key points. By determining the priority of input formats according to the parent's emotion, input efficiency is improved. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the format unit may be performed using AI, or may be performed without using AI. For example, the format unit can input the parent's emotion data into a generative AI and have the generative AI execute emotion estimation. Specifically, the format unit inputs text data entered by the parent (e.g., natural language sentences such as “I'm in a hurry today” or “Only the important items are needed”), audio data (e.g., audio waveform data, mel spectrograms), and facial image data (e.g., facial expression image tensors, shape: 224×224×3) into an emotion estimation AI. Examples of input to the AI include: (1) text sequences such as “Only the important items are needed” (integer array up to 512 tokens); (2) mel spectrograms of the parent's speech (shape: 128×256); and (3) facial expression image tensors (shape: 224×224×3). The format unit normalizes and extracts features from these input data in a preprocessing unit and inputs them into a multimodal emotion estimation AI integrating CNN and Transformer. The AI classifies emotion categories (e.g., stress, relaxation, hurry) from the input data and generates as output emotion labels (e.g., “in a hurry”), emotion scores (continuous values from 0.0 to 1.0), and text explaining the basis for emotion estimation (e.g., “Judged as in a hurry because the input sentence contains ‘in a hurry’”). Examples of AI output include: (1) emotion label “in a hurry”; (2) emotion score 0.91; and (3) explanation “The input sentence contains ‘in a hurry’”. Based on these outputs, the format unit instructs the input format priority determination module, for example, to display only important items first for the “stress” label, to sequentially display detailed items for the “relaxation” label, and to display only key points in bullet points for the “in a hurry” label, thereby automatically optimizing the UI. Furthermore, when the parent's emotional state changes, the format unit reconfigures the order of input formats in real time, always providing the optimal input experience. As a technical effect, the format unit achieves high-precision emotion estimation and dynamic optimization of input formats by AI, without relying on human subjective judgment or static input format design, thereby improving input efficiency, reducing input errors, and enhancing user satisfaction. Specific application fields include emotion-adaptive input format presentation for infant health checkup apps, stress-responsive input optimization in home health management systems, and emotion-linked automatic generation of electronic medical questionnaires for medical institutions.

The format unit can select the optimal format by considering the parent's geographic location information when creating the input format. For example, if the parent lives in a specific region, the format unit selects the format optimal for that region. Additionally, if the parent is traveling, the format unit can select the format optimal for the travel destination. Furthermore, if the parent is planning to move, the format unit can select the format optimal for the new region. By selecting the optimal format based on the parent's geographic location information, input efficiency is improved. Some or all of the above-described processing in the format unit may be performed using AI, or may be performed without using AI. For example, the format unit can input the parent's geographic location information data into a generative AI and have the generative AI execute selection of the optimal format. Specifically, the format unit normalizes GPS coordinate data obtained from the parent's device (e.g., numerical vectors for latitude and longitude, e.g., 35.6895, 139.6917), region codes linked to location information (e.g., prefecture, city/ward/town/village ID), and movement history (e.g., time-series vectors of location information for the past week, shape: 7×2) in a preprocessing unit and inputs them into the AI. Examples of input to the AI include: (1) “Shinjuku-ku, Tokyo, current location: 35.6938, 139.7034”; (2) “Travel destination: Kita-ku, Osaka, movement history: Tokyo→Osaka”; and (3) “Planned relocation: Nishi-ku, Yokohama” as structured data. The format unit inputs these geographic information into a Transformer-based location information analysis AI, which infers region-specific input formats (e.g., region-specific health items, region-specific lifestyle items, climate-adapted input items, etc.). The AI calculates relevance scores from the input geographic information and generates as output a list of optimal formats (e.g., “add pollen allergy countermeasure input field”, “add heatstroke risk input field”), priority for each format (e.g., importance score 0.92), and region-specific advice text (e.g., “Pollen allergy is prevalent in this region in spring”). Examples of AI output include: (1) format “add pollen allergy countermeasure input field”; (2) priority “pollen allergy countermeasure: 0.92, heatstroke risk: 0.85”; and (3) advice “Be careful of pollen allergy in spring”. Based on these outputs, the format unit automatically generates the input form and adds, deletes, or reorders items in real time according to the parent's current location or movement plans. Furthermore, when the parent's location information changes, the format unit immediately reconfigures the input format, always providing the optimal input experience. As a technical effect, the format unit automates dynamic optimization of input formats according to the user's geographic situation using AI, which is difficult with manual customization or static input format design, thereby improving input efficiency, standardizing data quality, and enhancing user satisfaction. The system can flexibly respond to region-specific health risks and lifestyle differences that could not be addressed by conventional uniform input forms, thereby improving the accuracy of growth data collection and greatly enhancing the reliability of subsequent AI analysis and referral letter creation. Specific application fields include region-adaptive input format generation for infant health checkup apps, region-specific health monitoring in home health management systems, and region-specific automatic generation of medical questionnaires for medical institutions.

The algorithm unit can estimate the parent's emotion and adjust the parameters of the analysis algorithm based on the estimated emotion. For example, if the parent is nervous, the algorithm unit provides a simple and highly visible algorithm. If the parent is relaxed, the algorithm unit can provide an algorithm that includes detailed information. Furthermore, if the parent is in a hurry, the algorithm unit can provide an algorithm that focuses on key points. By adjusting the parameters of the analysis algorithm according to the parent's emotion, the accuracy of the analysis is improved. Emotion estimation is realized, for example, by using an emotion engine or a generative AI with emotion estimation functionality. The generative AI may be a text-generating AI (e.g., LLM) or a multimodal generative AI, but is not limited to these examples. Some or all of the above-described processing in the algorithm unit may be performed using AI, or may be performed without using AI. For example, the algorithm unit can input the parent's emotion data into a generative AI and have the generative AI execute emotion estimation. Specifically, the algorithm unit inputs text data entered by the parent (e.g., natural language sentences such as “I'm nervous today” or “No need for detailed explanation”), audio data (e.g., audio waveform data, mel spectrograms), and facial image data (e.g., facial expression image tensors, shape: 224×224×3) into an emotion estimation AI. The algorithm unit normalizes and extracts features from these data in a preprocessing unit and inputs them into a multimodal emotion estimation AI integrating CNN and Transformer. Examples of input to the AI include: (1) text sequences such as “I'm nervous today” (up to 512 tokens); (2) mel spectrograms of the parent's speech (shape: 128×256); and (3) facial expression image tensors (shape: 224×224×3). The algorithm unit receives as output from the AI emotion labels (e.g., “nervous”, “relaxed”, “in a hurry”), emotion scores (continuous values from 0.0 to 1.0), and text explaining the basis for estimation (e.g., “Speech rate is fast and the input sentence contains ‘nervous’, so judged as nervous”). Based on these outputs, the algorithm unit instructs the analysis algorithm parameter adjustment module, for example, to relax thresholds, simplify explanations, and omit graphs or detailed outputs for the “nervous” label; to increase detailed analysis parameters (e.g., number of segments, number of features) and display detailed grounds for anomaly detection and statistical backing for the “relaxed” label; and to select algorithms that extract only key points (e.g., calculate only main abnormality scores) and reduce overall computational load for the “in a hurry” label. The algorithm unit re-adjusts parameters in real time when the parent's emotional state changes, always maintaining the optimal analysis experience. Examples of input to the AI include: (1) text such as “No need for details”; (2) spectrograms of the parent's speech; and (3) facial expression image tensors. Examples of AI output include: (1) emotion label “nervous”; (2) emotion score 0.85; and (3) explanation “The input sentence contains ‘nervous’”. Based on these outputs, the algorithm unit automatically selects analysis algorithm templates and adjusts parameters, providing analysis algorithms optimized for the parent's psychological state. As a technical effect, the algorithm unit achieves high-precision emotion estimation and dynamic parameter optimization by AI, without relying on human subjective judgment or static algorithm design, thereby improving understanding of analysis content, preventing confusion due to information overload, and enhancing satisfaction through user-specific optimization. The system enables flexible response to individual information needs, which was difficult with conventional uniform algorithms, and contributes to strengthened accountability and improved analysis accuracy in medical settings. Specific application fields include emotion-adaptive analysis algorithm control for infant health checkup apps, stress-responsive analysis parameter optimization in home health management systems, and automated personalized adjustment of analysis algorithms for medical institutions.

The algorithm unit can refer to past analysis data when creating the algorithm to generate the optimal algorithm. For example, the algorithm unit generates the optimal algorithm based on past analysis data. Additionally, the algorithm unit can analyze past analysis data to adjust the parameters of the algorithm. Furthermore, the algorithm unit can improve the accuracy of the algorithm by referring to past analysis data. By referring to past analysis data, the accuracy of the analysis algorithm is improved. Some or all of the above-described processing in the algorithm unit may be performed using AI, or may be performed without using AI. For example, the algorithm unit can input past analysis data into a generative AI and have the generative AI execute generation of the optimal algorithm. Specifically, the algorithm unit inputs a past analysis database (e.g., abnormality detection history, judgment scores, input data features, types of analysis algorithms, parameter sets, accuracy of analysis results, and cases of misjudgment as structured data) as time-series vectors (shape: N×M, where N is the number of data entries and M is the number of features), text sequences (up to 2048 tokens), and image data (analysis target images and graphs, shape: 224×224×3) into the AI. Examples of input to the AI include: (1) analysis history vectors for the past year (e.g., date, algorithm ID, judgment score, accuracy); (2) text records of misjudgment cases; and (3) analysis target image data. The algorithm unit inputs these data into the AI using supervised learning, transfer learning, or self-supervised learning methods, and the AI automatically selects the optimal algorithm from multiple algorithms such as CNN, Transformer, decision tree, and random forest. The AI performs cross-validation and grid search on the input data and generates as output the name of the optimal algorithm (e.g., “LSTM+Attention”), parameter set (e.g., “learning rate=0.001, batch size=32”), and accuracy evaluation score (e.g., AUC=0.94, F1=0.91). Examples of AI output include: (1) optimal algorithm “LSTM+Attention”; (2) parameters “learning rate 0.001, batch size 32”; and (3) accuracy evaluation “AUC=0.94”. Based on these outputs, the algorithm unit automatically configures the analysis pipeline, strengthening reproducibility, generalizability, and the ability to discover new abnormality patterns in analysis results. Furthermore, the algorithm unit continuously readjusts parameters based on feedback from analysis results to improve model accuracy. As a technical effect, the algorithm unit realizes automatic design and optimization of the optimal AI analysis pipeline according to data characteristics and analysis objectives, which is difficult with manual algorithm selection or static rule-based processing, thereby greatly improving analysis accuracy, reproducibility, and generalizability. This enables individualized analysis support, early detection of misjudgment patterns, and standardization of analysis methods in medical settings. Specific application fields include automatic analysis optimization for infant health checkup apps, medical big data analysis platforms, and personalized optimization of analysis algorithms for medical institutions.

The algorithm unit can estimate the parent's emotion and determine the priority of analysis algorithms based on the estimated emotion. For example, when the parent is feeling stressed, the algorithm unit preferentially provides the most important analysis algorithms. When the parent is relaxed, the algorithm unit can sequentially provide detailed analysis algorithms. Furthermore, when the parent is in a hurry, the algorithm unit can preferentially provide analysis algorithms that focus on key points. By determining the priority of analysis algorithms according to the parent's emotion, important analysis results can be provided preferentially. Emotion estimation is realized, for example, by using an emotion engine or a generative AI with emotion estimation functionality. The generative AI may be a text generation AI (such as an LLM) or a multimodal generative AI, but is not limited to these examples. Some or all of the above-described processing in the algorithm unit may be performed using AI or without using AI. For example, the algorithm unit can input the parent's emotion data to the generative AI and have the generative AI perform emotion estimation. Specifically, the algorithm unit inputs to the emotion estimation AI the text data entered by the parent (e.g., natural language sentences such as “I am very stressed today” or “I only want to know the important results”), audio data (e.g., audio waveform data, Mel spectrogram), and facial image data (e.g., facial expression image tensor, shape: 224×224×3). The algorithm unit normalizes and extracts features from these data in a preprocessing unit and inputs them to a multimodal emotion estimation AI integrating CNN and Transformer. Examples of input to the AI include: (1) text sequences such as “I only want to know the important results” (up to 512 tokens); (2) Mel spectrogram of the parent's speech (shape: 128×256); and (3) facial expression image tensor of the parent (shape: 224×224×3). The algorithm unit receives from the AI output such as emotion labels (e.g., “stressed”, “relaxed”, “in a hurry”), emotion scores (continuous values from 0.0 to 1.0), and explanation text for the estimation basis (e.g., “Determined as stressed because the word ‘important’ is included in the input sentence”). Based on these outputs, the algorithm unit instructs the analysis algorithm priority determination module, for example, to execute anomaly detection algorithms or key item extraction algorithms first when the label is “stressed”, and postpone detailed statistical analysis or graph generation. When the label is “relaxed”, detailed analysis algorithms (e.g., multivariate analysis, time series analysis) are executed sequentially, and explanations and bases for the analysis results are presented in detail. When the label is “in a hurry”, key point extraction algorithms are prioritized and overall computational load is reduced. The algorithm unit reconstructs the priority in real time when the parent's emotional state changes, always maintaining an optimal analysis experience. Examples of input to the AI include: (1) text such as “Just the key points today”; (2) spectrogram of the parent's speech; and (3) facial expression image tensor. Examples of output from the AI include: (1) emotion label “in a hurry”; (2) emotion score 0.91; and (3) explanation “The word ‘key points’ is included in the input sentence”. The algorithm unit automatically adjusts the execution order and output content of analysis algorithms based on these outputs, providing analysis results optimized for the parent's psychological state. As a technical effect, the algorithm unit achieves high-precision emotion estimation and dynamic priority control by AI, without relying on human subjective judgment or static algorithm execution order design, thereby preventing the oversight of important information, improving user satisfaction, and enhancing the efficiency of analysis operations. Flexible response to individual user information needs, which was difficult with conventional uniform analysis algorithms, becomes possible, contributing to strengthened accountability in medical settings and improved analysis accuracy. Specific application fields include emotion-adaptive analysis algorithm execution in infant health checkup apps, stress-responsive analysis prioritization in home health management systems, and personalized execution order control of analysis algorithms for medical institutions.

The algorithm unit can select the optimal algorithm by considering the parent's geographic location information at the time of algorithm creation. For example, if the parent lives in a specific region, the algorithm unit selects an algorithm optimal for that region. If the parent is traveling, the algorithm unit can select an algorithm optimal for the travel destination. Furthermore, if the parent is planning to move, the algorithm unit can select an algorithm optimal for the new region. By selecting the optimal algorithm based on the parent's geographic location information, the accuracy of analysis is improved. Some or all of the above-described processing in the algorithm unit may be performed using AI or without using AI. For example, the algorithm unit can input the parent's geographic location information data to the generative AI and have the generative AI select the optimal algorithm. Specifically, the algorithm unit normalizes GPS coordinate data obtained from the parent's device (e.g., numerical vectors of latitude and longitude, such as 35.6895, 139.6917), region codes linked to location information (e.g., prefecture, city/ward/town/village ID), and movement history (e.g., time series vector of location information for the past week, shape: 7×2) in a preprocessing unit and inputs them to the AI. Examples of input to the AI include: (1) “Shinjuku-ku, Tokyo, current location: 35.6938, 139.7034”; (2) “Travel destination: Kita-ku, Osaka, movement history: Tokyo→Osaka”; and (3) “Planned move: Nishi-ku, Yokohama” as structured data. The algorithm unit inputs this geographic information to a Transformer-based location information analysis AI, which automatically selects analysis algorithms considering region-specific health risks, medical resources, lifestyle habits, climate conditions, etc. (e.g., pollen allergy risk assessment, heatstroke risk assessment, application of region-specific growth curves). The AI calculates relevance scores from the input geographic information and outputs an optimal algorithm list (e.g., “pollen allergy risk assessment algorithm”, “heatstroke risk assessment algorithm”), priority for each algorithm (e.g., importance score 0.92), and selection reason text (e.g., “This region frequently experiences pollen allergy in early spring”). Examples of output from the AI include: (1) algorithm “pollen allergy risk assessment”; (2) priority “pollen allergy: 0.92, heatstroke: 0.85”; and (3) reason “Be cautious of pollen allergy in early spring”. Based on these outputs, the algorithm unit automatically generates an analysis pipeline and adds, removes, or changes the order of algorithms in real time according to the parent's current location or planned movement. Furthermore, when the parent's location information changes, the algorithm unit immediately reconstructs the algorithm selection, always providing an optimal analysis experience. As a technical effect, the algorithm unit automates dynamic optimization of analysis algorithms according to the user's geographic situation using AI, which is difficult with manual customization or static algorithm design, thereby improving the efficiency of analysis operations, homogenizing analysis quality, and enhancing user satisfaction. Flexible response to region-specific health risks and lifestyle differences, which could not be addressed by conventional uniform analysis algorithms, becomes possible, improving the accuracy of growth data analysis and greatly enhancing the reliability of subsequent referral letter creation and medical support. Specific application fields include region-adaptive analysis algorithm generation in infant health checkup apps, region-specific health risk analysis in home health management systems, and region-specialized automatic generation of analysis algorithms for medical institutions.

The system according to the embodiment is not limited to the above-described examples and can be variously modified as follows. Specifically, the system can flexibly change the configuration of each module such as the analysis unit, creation unit, distribution unit, learning unit, format unit, and algorithm unit. For example, in the analysis unit, a hybrid analysis pipeline combining image analysis AI and time series analysis AI can be introduced. In the creation unit, multiple automatic referral letter generation templates can be prepared, and the template can be dynamically switched according to the parent's emotion and living situation. In the distribution unit, multiple distribution means such as email, app notifications, cloud storage, and postal mail can be combined, and the optimal distribution method can be automatically selected according to the parent's device usage and communication environment. In the learning unit, various learning methods such as supervised learning, transfer learning, and self-supervised learning can be switched, and the optimal learning pipeline can be configured according to data characteristics and learning objectives. In the format unit, the layout of input forms, order of items, color scheme, and input methods (text, voice, image, etc.) can be dynamically optimized according to the parent's emotion and input history. In the algorithm unit, the types of analysis algorithms and parameter sets can be automatically generated and optimized based on the parent's emotion, geographic information, and past analysis history. Furthermore, the system can utilize a distributed processing platform on the cloud or a parallel computing cluster using GPUs to support real-time analysis of large-scale data and simultaneous processing for multiple users. As a technical effect, the system automates dynamic optimization according to each user's situation, emotion, region, and history using AI, which was difficult with conventional static system design and manual operation, thereby greatly improving analysis accuracy, operational efficiency, user satisfaction, and system scalability. Specific application fields include personalized analysis, referral letter generation, and distribution support in infant health checkup apps, business automation platforms for medical institutions, and individualized optimization support in home health management systems.

The reception unit can provide real-time feedback on growth data entered by the parent, prompting confirmation or correction of the input content. For example, when the parent enters height or weight, the system immediately displays whether there are any abnormalities compared to past data and prompts re-entry if necessary. Additionally, the reception unit can automatically generate related questions based on the input content to supplement information that the parent may overlook. Furthermore, the reception unit can display appropriate advice or points of caution according to the input content, supporting the parent to complete the input with peace of mind. As a result, the accuracy of growth data entered by the parent is improved, and the overall reliability of the system is enhanced.

The learning unit can not only specify the type and scope of data to be learned by the generative AI, but also evaluate the quality of the data and exclude low-quality data. For example, the learning unit evaluates the resolution of image data and the consistency of text data, and automatically filters out data that do not meet the criteria. Additionally, the learning unit evaluates the source and reliability of the data and can exclude data with low reliability. Furthermore, the learning unit evaluates the recency of the data and appropriately updates old data. As a result, by having the generative AI learn high-quality data, the accuracy of analysis is further improved.

The format unit can not only specify the format of information input by the parent, but also automatically optimize the format based on the input content. For example, the layout and order of items in the input form can be dynamically changed according to the type and amount of data entered by the parent. Additionally, the format unit can adjust the input interface according to the parent's input speed and input method (such as text or voice). Furthermore, when an error is detected during input, the format unit can display a message prompting real-time correction, thereby improving the accuracy of input. As a result, the parent can efficiently and accurately input information.

The algorithm unit can not only specify the algorithm to be used by the generative AI during analysis, but also automatically improve the algorithm based on the analysis results. For example, the algorithm unit evaluates the accuracy and reliability of the analysis results and adjusts the algorithm parameters as necessary. Additionally, the algorithm unit receives feedback on the analysis results and continuously optimizes the algorithm. Furthermore, the algorithm unit can combine different analysis methods to generate hybrid algorithms, thereby improving the accuracy of analysis. As a result, the generative AI can perform more accurate and reliable analysis.

The reception unit can not only estimate the parent's emotion and adjust the design of the input interface based on the estimated emotion, but also dynamically change the priority of input content according to the parent's emotion. For example, when the parent is feeling stressed, the most important input items are preferentially displayed, and detailed input items are postponed. When the parent is relaxed, detailed input items are displayed sequentially, allowing the parent to enjoy the input process. Furthermore, when the parent is in a hurry, voice input is prioritized to enable quick input. As a result, input efficiency is improved according to the parent's emotion, and the parent's burden is reduced.

The reception unit can not only analyze the parent's past input history and propose an optimal input method, but also learn the parent's input patterns and predict future inputs. For example, based on growth data that the parent has frequently entered in the past, the reception unit predicts the content of the next input and automatically displays it as a candidate. Additionally, the reception unit analyzes the parent's input frequency and time of day, and can propose the optimal timing for input. Furthermore, the reception unit learns the parent's input style (such as voice or text) and can continuously provide the optimal input method for the parent. As a result, the parent's input work is streamlined and the usability of the system is improved.

The reception unit can not only customize input items at the time of input based on the parent's current living situation and areas of interest, but also dynamically update input items according to changes in the parent's lifestyle. For example, when the parent starts a new job, the system prompts input of growth data considering the impact of the job. When the parent starts a new hobby, the system can propose input of growth data related to that hobby. Furthermore, when the parent moves, the system can prompt input of growth data according to the environment of the new region. As a result, by flexibly customizing input items according to the parent's lifestyle, input efficiency is improved.

The reception unit can not only estimate the parent's emotion and determine the priority of input based on the estimated emotion, but also provide feedback on the input content according to the parent's emotion. For example, when the parent is feeling stressed, the system provides positive feedback on the input content to enhance the parent's sense of security. When the parent is relaxed, the system provides detailed feedback to help the parent deeply understand the input content. Furthermore, when the parent is in a hurry, the system provides concise and focused feedback to enable the parent to complete the input work quickly. As a result, by providing appropriate feedback according to the parent's emotion, input efficiency is improved and parent satisfaction is increased.

The reception unit can not only preferentially display highly relevant input items at the time of input by considering the parent's geographic location information, but also provide region-specific advice and information based on the parent's location information. For example, when the parent lives in a specific region, the system prompts input of growth data according to the climate and environment of that region. When the parent is traveling, the system can provide information on medical facilities and emergency contacts at the travel destination. Furthermore, when the parent is planning to move, the system can provide medical information and child-rearing support information for the new region. As a result, by not only displaying input items based on the parent's geographic location information but also providing region-specific information, the parent's sense of security is enhanced.

The reception unit can not only analyze the parent's social media activity at the time of input and propose relevant input items, but also automatically supplement input content based on the parent's social media activity. For example, growth data about the child shared by the parent on social media can be automatically reflected in the input form. Additionally, information obtained from health-related accounts followed by the parent on social media can be used to supplement input content. Furthermore, information from parenting groups in which the parent participates on social media can be used to automatically add relevant input items. As a result, by not only analyzing the parent's social media activity but also automatically supplementing input content, the input work is streamlined.

The following is a brief explanation of the processing flow of Example of the Embodiment.
Step 1: The reception unit receives input from the parent regarding the growth status of the child. The parent can input information such as height, weight, tooth eruption, and walking behavior. The reception unit can receive information by methods such as text input or voice input.
Step 2: The analysis unit analyzes the information input by the reception unit using a generative AI. The generative AI has learned past growth data of children and image data such as tooth alignment, and checks whether there are any abnormalities by comparing with the input information. For example, it detects cases where height or weight deviates significantly from the average value or where abnormalities are observed in tooth alignment.
Step 3: The creation unit creates a referral letter when an abnormality is detected by the analysis unit. The referral letter describes the growth status of the child and the details of the abnormality.
Step 4: The distribution unit distributes the referral letter created by the creation unit in PDF format. By bringing this referral letter to the hospital, the parent can receive prompt and appropriate medical examination.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, analysis unit, creation unit, and distribution unit is implemented by at least one of, for example, a smart device 14 and a data processing apparatus 12. For example, the reception unit is implemented by a control unit 46A of the smart device 14, and the parent inputs the growth status of the child using the smart device 14. The analysis unit is implemented, for example, by a specific processing unit 290 of the data processing apparatus 12, and analyzes the input information using generative AI. The creation unit is implemented, for example, by the specific processing unit 290 of the data processing apparatus 12, and creates a referral letter when an abnormality is detected. The distribution unit is implemented, for example, by the control unit 46A of the smart device 14, and distributes the created referral letter in PDF format. The correspondence between each unit and the apparatus or control unit is not limited to the examples described above, and various modifications are possible.

### Second Embodiment

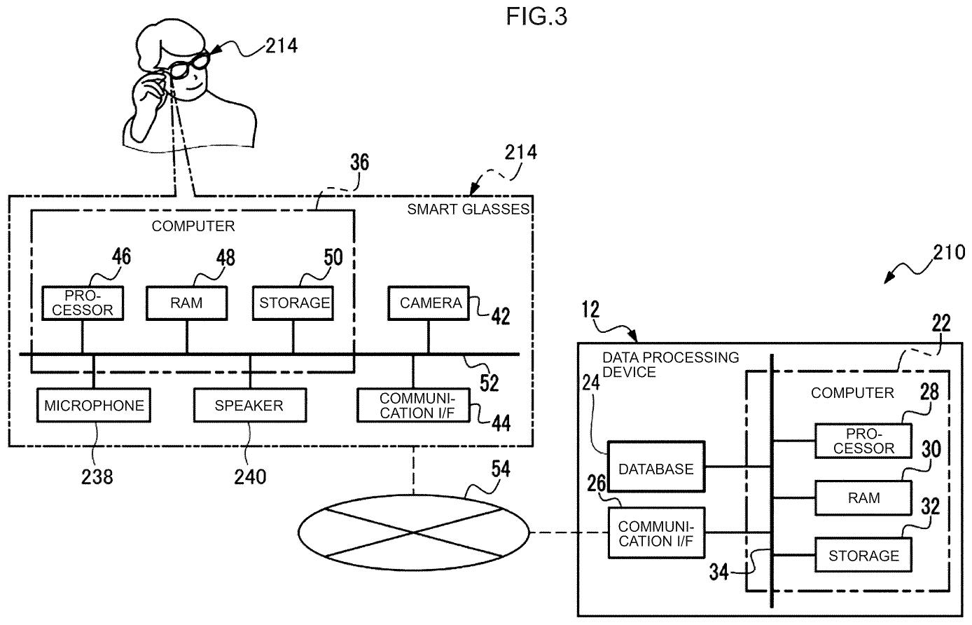

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

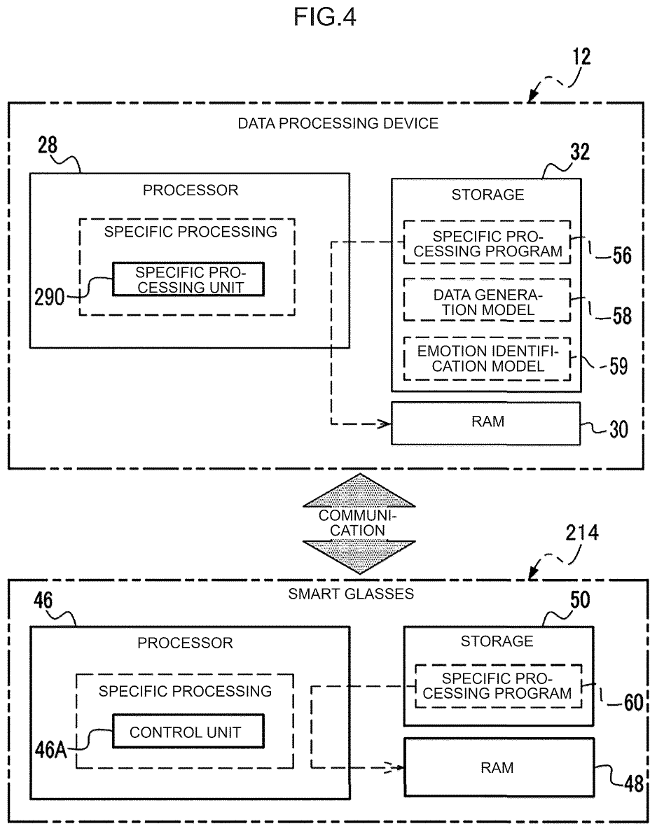

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, analysis unit, creation unit, and distribution unit is implemented by at least one of, for example, smart glasses 214 and a data processing apparatus 12. For example, the reception unit is implemented by a control unit 46A of the smart glasses 214, and the parent inputs the growth status of the child using the smart glasses 214. The analysis unit is implemented, for example, by a specific processing unit 290 of the data processing apparatus 12, and analyzes the input information using generative AI. The creation unit is implemented, for example, by the specific processing unit 290 of the data processing apparatus 12, and creates a referral letter when an abnormality is detected. The distribution unit is implemented, for example, by the control unit 46A of the smart glasses 214, and distributes the created referral letter in PDF format. The correspondence between each unit and the apparatus or control unit is not limited to the examples described above, and various modifications are possible.

### Third Embodiment

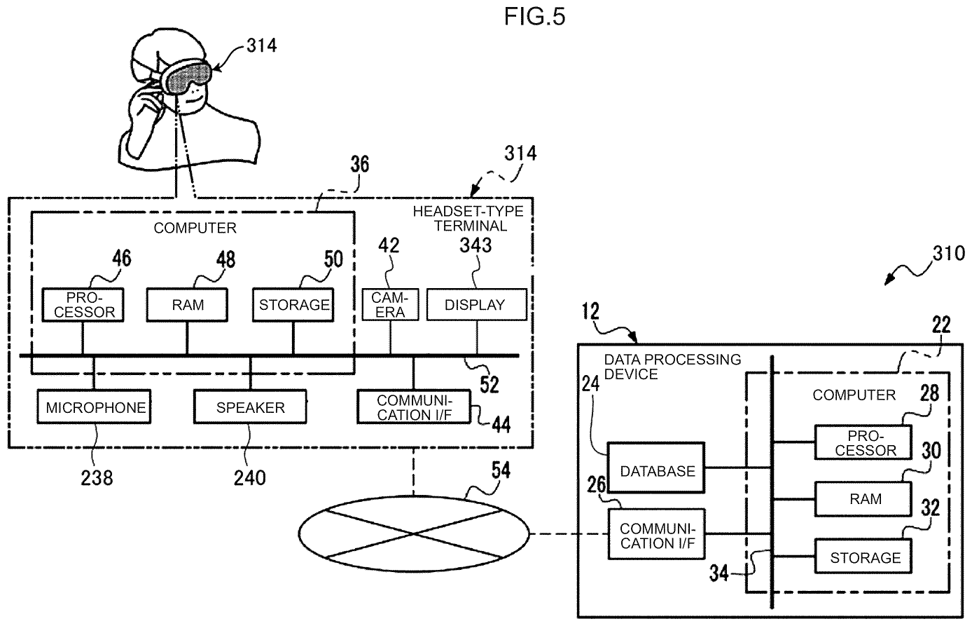

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

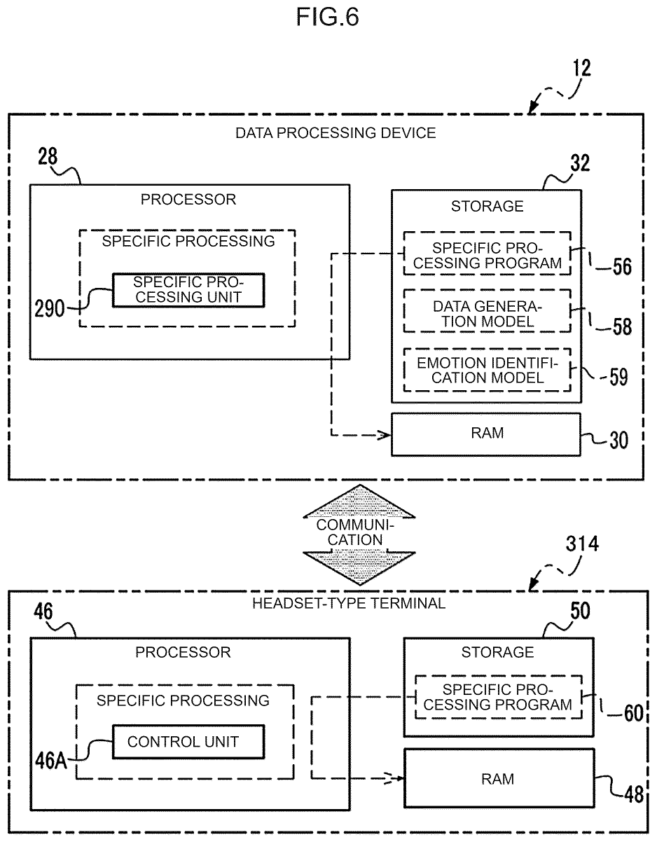

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, analysis unit, creation unit, and distribution unit is implemented by at least one of, for example, a headset-type terminal 314 and a data processing apparatus 12. For example, the reception unit is implemented by a control unit 46A of the headset-type terminal 314, and the parent inputs the growth status of the child using the headset-type terminal 314. The analysis unit is implemented, for example, by a specific processing unit 290 of the data processing apparatus 12, and analyzes the input information using generative AI. The creation unit is implemented, for example, by the specific processing unit 290 of the data processing apparatus 12, and creates a referral letter when an abnormality is detected. The distribution unit is implemented, for example, by the control unit 46A of the headset-type terminal 314, and distributes the created referral letter in PDF format. The correspondence between each unit and the apparatus or control unit is not limited to the examples described above, and various modifications are possible.

### Fourth Embodiment

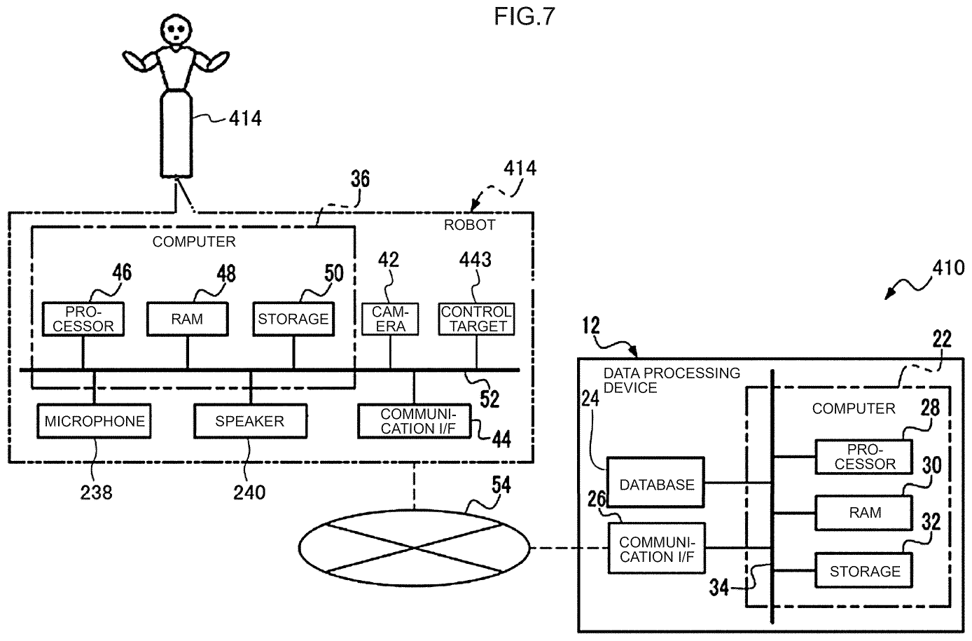

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

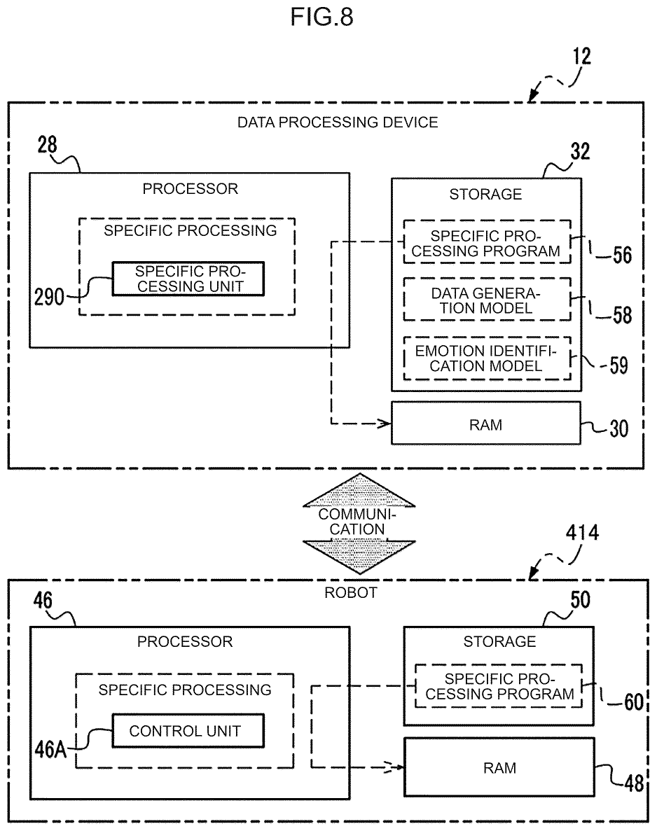

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, analysis unit, creation unit, and distribution unit is implemented by at least one of, for example, a robot 414 and a data processing apparatus 12. For example, the reception unit is implemented by a control unit 46A of the robot 414, and the parent inputs the growth status of the child using the robot 414. The analysis unit is implemented, for example, by a specific processing unit 290 of the data processing apparatus 12, and analyzes the input information using generative AI. The creation unit is implemented, for example, by the specific processing unit 290 of the data processing apparatus 12, and creates a referral letter when an abnormality is detected. The distribution unit is implemented, for example, by the control unit 46A of the robot 414, and distributes the created referral letter in PDF format. The correspondence between each unit and the apparatus or control unit is not limited to the examples described above, and various modifications are possible.

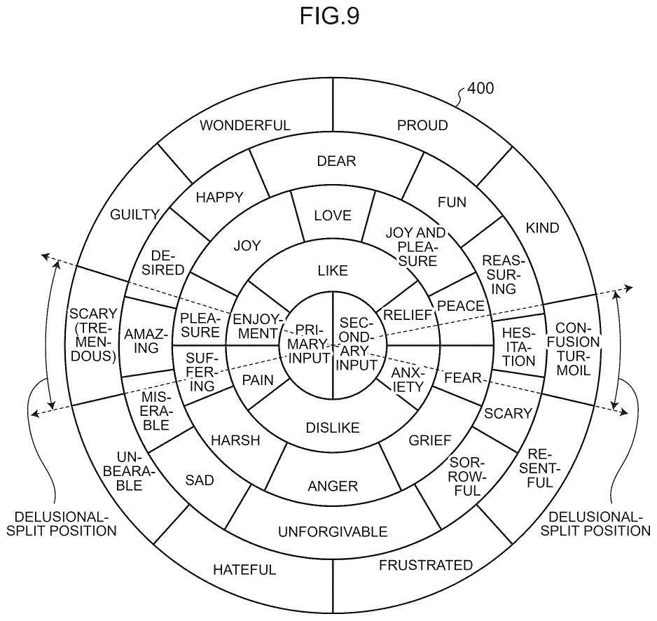

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

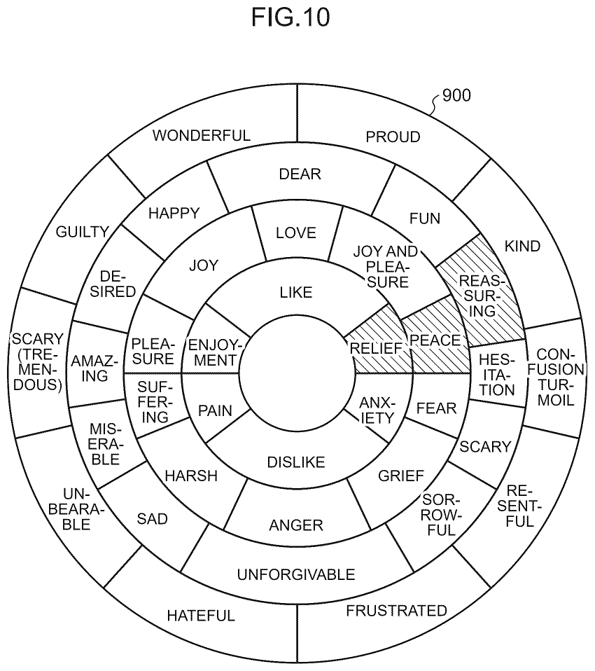

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a reception unit configured to receive input from a parent regarding the growth status of a child; an analysis unit configured to analyze information input by the reception unit; a creation unit configured to create a referral letter when an abnormality is detected by the analysis unit; and a distribution unit configured to distribute the referral letter created by the creation unit in PDF format.
(Supplementary Note 2) The system according to Supplementary Note 1, further comprising a learning unit configured to specify the type and scope of data to be learned by a generative AI.
(Supplementary Note 3) The system according to Supplementary Note 1, further comprising a format unit configured to specify the format of information input by the parent.
(Supplementary Note 4) The system according to Supplementary Note 1, further comprising an algorithm unit configured to specify an algorithm to be used by the generative AI during analysis.
(Supplementary Note 5) The system according to Supplementary Note 1, wherein the reception unit is configured to estimate the parent's emotion and adjust the design of the input interface based on the estimated emotion.
(Supplementary Note 6) The system according to Supplementary Note 1, wherein the reception unit is configured to analyze the parent's past input history and propose an appropriate input method.
(Supplementary Note 7) The system according to Supplementary Note 1, wherein the reception unit is configured to customize input items at the time of input based on the parent's current living situation and areas of interest.
(Supplementary Note 8) The system according to Supplementary Note 1, wherein the reception unit is configured to estimate the parent's emotion and determine the priority of input based on the estimated emotion.
(Supplementary Note 9) The system according to Supplementary Note 1, wherein the reception unit is configured to preferentially display highly relevant input items at the time of input by considering the parent's geographic location information.
(Supplementary Note 10) The system according to Supplementary Note 1, wherein the reception unit is configured to analyze the parent's social media activity at the time of input and propose relevant input items.
(Supplementary Note 11) The system according to Supplementary Note 1, wherein the analysis unit is configured to estimate the parent's emotion and adjust the display method of the analysis result based on the estimated emotion.
(Supplementary Note 12) The system according to Supplementary Note 1, wherein the analysis unit is configured to optimize the analysis algorithm by referring to past growth data during analysis.
(Supplementary Note 13) The system according to Supplementary Note 1, wherein the analysis unit is configured to apply different analysis methods according to the category of the child during analysis.
(Supplementary Note 14) The system according to Supplementary Note 1, wherein the analysis unit is configured to estimate the parent's emotion and determine the priority of the analysis result based on the estimated emotion.
(Supplementary Note 15) The system according to Supplementary Note 1, wherein the analysis unit is configured to weight the analysis based on the submission timing of the child's growth data during analysis.
(Supplementary Note 16) The system according to Supplementary Note 1, wherein the analysis unit is configured to improve the accuracy of analysis by referring to relevant medical literature during analysis.
(Supplementary Note 17) The system according to Supplementary Note 1, wherein the creation unit is configured to estimate the parent's emotion and adjust the content of the referral letter based on the estimated emotion.
(Supplementary Note 18) The system according to Supplementary Note 1, wherein the creation unit is configured to generate appropriate content by referring to past referral letter data when creating the referral letter.
(Supplementary Note 19) The system according to Supplementary Note 1, wherein the creation unit is configured to customize the format of the referral letter based on the child's growth data when creating the referral letter.
(Supplementary Note 20) The system according to Supplementary Note 1, wherein the creation unit is configured to estimate the parent's emotion and determine the priority of the referral letter based on the estimated emotion.
(Supplementary Note 21) The system according to Supplementary Note 1, wherein the creation unit is configured to select an optimal hospital by considering the child's geographic location information when creating the referral letter.
(Supplementary Note 22) The system according to Supplementary Note 1, wherein the creation unit is configured to enrich the content of the referral letter by referring to relevant medical literature when creating the referral letter.
(Supplementary Note 23) The system according to Supplementary Note 1, wherein the distribution unit is configured to estimate the parent's emotion and adjust the distribution method of the referral letter based on the estimated emotion.
(Supplementary Note 24) The system according to Supplementary Note 1, wherein the distribution unit is configured to select an optimal distribution method by referring to the parent's past distribution history when distributing the referral letter.
(Supplementary Note 25) The system according to Supplementary Note 1, wherein the distribution unit is configured to customize the distribution means based on the parent's current living situation when distributing the referral letter.
(Supplementary Note 26) The system according to Supplementary Note 1, wherein the distribution unit is configured to estimate the parent's emotion and determine the priority of distribution of the referral letter based on the estimated emotion.
(Supplementary Note 27) The system according to Supplementary Note 1, wherein the distribution unit is configured to select an optimal distribution means by considering the parent's geographic location information when distributing the referral letter.
(Supplementary Note 28) The system according to Supplementary Note 1, wherein the distribution unit is configured to analyze the parent's social media activity and propose distribution means when distributing the referral letter.
(Supplementary Note 29) The system according to Supplementary Note 2, wherein the learning unit is configured to estimate the parent's emotion and select learning data based on the estimated emotion.
(Supplementary Note 30) The system according to Supplementary Note 2, wherein the learning unit is configured to optimize the learning algorithm by referring to past learning data during learning.
(Supplementary Note 31) The system according to Supplementary Note 2, wherein the learning unit is configured to estimate the parent's emotion and adjust the frequency of learning based on the estimated emotion.
(Supplementary Note 32) The system according to Supplementary Note 2, wherein the learning unit is configured to weight learning data based on the submission timing of growth data during learning.
(Supplementary Note 33) The system according to Supplementary Note 3, wherein the format unit is configured to estimate the parent's emotion and adjust the design of the input format based on the estimated emotion.
(Supplementary Note 34) The system according to Supplementary Note 3, wherein the format unit is configured to generate an optimal format by referring to past input data when creating the input format.
(Supplementary Note 35) The system according to Supplementary Note 3, wherein the format unit is configured to estimate the parent's emotion and determine the priority of the input format based on the estimated emotion.
(Supplementary Note 36) The system according to Supplementary Note 3, wherein the format unit is configured to select an optimal format by considering the parent's geographic location information when creating the input format.
(Supplementary Note 37) The system according to Supplementary Note 4, wherein the algorithm unit is configured to estimate the parent's emotion and adjust parameters of the analysis algorithm based on the estimated emotion.
(Supplementary Note 38) The system according to Supplementary Note 4, wherein the algorithm unit is configured to generate an optimal algorithm by referring to past analysis data when creating the algorithm.
(Supplementary Note 39) The system according to Supplementary Note 4, wherein the algorithm unit is configured to estimate the parent's emotion and determine the priority of the analysis algorithm based on the estimated emotion.
(Supplementary Note 40) The system according to Supplementary Note 4, wherein the algorithm unit is configured to select an optimal algorithm by considering the parent's geographic location information when creating the algorithm.

What is claimed is:

1. A system comprising:
circuitry configured to:
receive, from a client terminal via a wireless transceiver communicatively coupled to a packet-switched network, structured data comprising a multidimensional feature vector;
compute an anomaly score by inputting the multidimensional feature vector into an anomaly detection model stored in a memory, the anomaly detection model comprising a neural network;
generate, based on the anomaly score exceeding a threshold, inference data by inputting the multidimensional feature vector and the anomaly score into a data generation model, the data generation model comprising a transformer-based language model; and
transmit, to the client terminal via the wireless transceiver, the inference data.

2. The system according to claim 1, wherein the structured data comprises biological data associated with a child, the biological data comprising at least one of height data, weight data, or dental alignment image data.

3. The system according to claim 1, wherein the inference data comprises a referral document formatted in a portable document format.

4. The system according to claim 1, wherein the circuitry is further configured to estimate an emotion of a user based on at least one of text data, voice data, or facial image data received from the client terminal, and adjust a design of an input interface transmitted to the client terminal based on the estimated emotion.

5. The system according to claim 4, wherein, when the estimated emotion indicates stress, the circuitry adjusts the input interface to display a reduced number of input items, and when the estimated emotion indicates relaxation, the circuitry adjusts the input interface to display detailed input options.

6. The system according to claim 1, wherein the circuitry is further configured to analyze a past input history associated with the user stored in a database, and propose an input method based on the past input history, the input method comprising at least one of voice input or text input.

7. The system according to claim 1, wherein the circuitry is further configured to customize input items based on at least one of a living situation or an area of interest associated with a user, such that when the area of interest comprises health, the circuitry generates input items comprising detailed health-related data fields.

8. The system according to claim 1, wherein the circuitry is further configured to estimate an emotion of a user and determine a priority of the input items based on the estimated emotion, such that when the estimated emotion indicates urgency, the circuitry preferentially displays high-priority input items.

9. The system according to claim 1, wherein the circuitry is further configured to receive geographic location information from the client terminal, and preferentially display input items relevant to a geographic region associated with the geographic location information.

10. The system according to claim 1, wherein the circuitry is further configured to analyze social media activity data associated with a user, and propose input items based on the social media activity data.

11. The system according to claim 1, wherein the anomaly detection model comprises a convolutional neural network configured to extract a feature map from image data included in the multidimensional feature vector, and detect the anomaly based on the feature map.

12. The system according to claim 1, wherein the circuitry is further configured to apply different analysis algorithms according to a category associated with the structured data, the category comprising at least one of age or gender.

13. The system according to claim 1, wherein the circuitry is further configured to estimate an emotion of a user and determine a priority of displaying analysis results based on the estimated emotion, such that when the estimated emotion indicates stress, the circuitry preferentially displays important analysis results.

14. The system according to claim 1, wherein the circuitry is further configured to weight the anomaly detection based on a submission timing of the structured data, such that recently submitted structured data is weighted more heavily than older structured data.

15. The system according to claim 1, wherein the circuitry is further configured to retrieve medical literature data from a database, and improve accuracy of the anomaly detection by incorporating the medical literature data into the anomaly detection model.

16. The system according to claim 1, wherein the circuitry is further configured to estimate an emotion of a user and adjust content of the inference data based on the estimated emotion, such that when the estimated emotion indicates nervousness, the circuitry generates the inference data in a simplified format.

17. The system according to claim 1, wherein the circuitry is further configured to receive geographic location information from the client terminal, and select a recommended facility based on the geographic location information for inclusion in the inference data.

18. A system comprising:
a communication interface comprising a wireless transceiver configured to communicate with a client terminal via a packet-switched network;
a processor;
a random-access memory coupled to the processor;
a memory storing an anomaly detection model comprising a convolutional neural network and a data generation model comprising a transformer-based language model;
a database configured to store reference data and user history data; and
circuitry configured to:
receive, from the client terminal via the communication interface, structured data comprising biological data associated with a child, the biological data comprising at least one of height data, weight data, dental eruption data, walking behavior data, or dental alignment image data;
preprocess the structured data to generate a multidimensional feature vector, the preprocessing comprising normalizing numerical data and converting image data into an image tensor;
compute an anomaly score by inputting the multidimensional feature vector into the anomaly detection model, wherein the anomaly detection model compares the multidimensional feature vector against the reference data stored in the database;
generate, based on the anomaly score exceeding a threshold, a referral document by inputting the multidimensional feature vector, the anomaly score, and user history data retrieved from the database into the data generation model;
format the referral document in a portable document format; and
transmit, to the client terminal via the communication interface, the referral document.

19. The system according to claim 18, wherein the circuitry is further configured to estimate an emotion of a user based on at least one of text data, voice data, or facial image data received from the client terminal by inputting the at least one of the text data, the voice data, or the facial image data into an emotion estimation model comprising an integrated convolutional neural network and transformer model, and adjust at least one of a design of an input interface, a priority of input items, or content of the referral document based on the estimated emotion.

20. A method performed by circuitry of a system, the method comprising:
receiving, from a client terminal via a wireless transceiver communicatively coupled to a packet-switched network, structured data comprising a multidimensional feature vector;
computing an anomaly score by inputting the multidimensional feature vector into an anomaly detection model stored in a memory, the anomaly detection model comprising a neural network;
generating, based on the anomaly score exceeding a threshold, inference data by inputting the multidimensional feature vector and the anomaly score into a data generation model, the data generation model comprising a transformer-based language model; and
transmitting, to the client terminal via the wireless transceiver, the inference data.
